SEQUENCE LISTING (33 sequences)

SEQ ID NO: 1 - DNA, 52 bp, Salmonella typhimurium
  source 1..52: mol_type = other DNA
tttaatttgtttgttagttagtttatttgttggtttgtttgtgttataatat

SEQ ID NO: 2 - DNA, 29 bp, Lactobacillus plantarum
  source 1..29: mol_type = genomic DNA
ttgacataaataccactggcggtgatact

SEQ ID NO: 3 - DNA, 28 bp, Lactobacillus plantarum
  source 1..28: mol_type = genomic DNA
ttgactattttacctctggcggtgataa

SEQ ID NO: 4 - DNA, 42 bp, Lactobacillus plantarum
  source 1..42: mol_type = genomic DNA
tctgtttacaaatcagattaggctatatataatatttaagga

SEQ ID NO: 5 - DNA, 48 bp, synthetic construct
  source 1..48: mol_type = other DNA
gcccatattaacgtttaaccgataaagttgaacgttaatatttttttt

SEQ ID NO: 6 - DNA, 40 bp, synthetic construct
  source 1..40: mol_type = other DNA
agttgttgacatggaacgaggaatgtgataatctgtgagt

SEQ ID NO: 7 - DNA, 54 bp, synthetic construct
  source 1..54: mol_type = other DNA
ctgatgacaaaaagagaaaattttgataaaatagtcttagaattaaattaaaaa

SEQ ID NO: 8 - DNA, 258 bp, Enterococcus faecium
  source 1..258: mol_type = genomic DNA
ttaatagtgatcttttgcagaataaataaatatcgtttcattttcaactctatatatcagtctatgttcatctgtaattcttctggaccattttccagataaatcatgctttaatggctcaggttttcctaatccagcaaagggggaacgatcgatatcttttattaacttgttaatcttttttatattgcttttgtttccttgctcatgccaataaagataatcatcccaagcatcatcagaccaagccttaatcat

SEQ ID NO: 9 - DNA, 270 bp, Enterococcus faecium
  source 1..270: mol_type = genomic DNA
ttaatcatcagattcaacctcgattaagtcatgtgttttaaatgcacctttggagaattgttcatctcctcgacgaattttttccatgacgtaattattagaaagtgttctcaacgtttcttgcatagaatcataatctctttttgataatacaacaactgtatcttctacatctttacttgttacaataagtgtttcagcatcctcattaacttgtttcatataactacgtaaattttggcggaaatttgaataagctactgcttccat

SEQ ID NO: 10 - DNA, 303 bp, Lactobacillus casei
  source 1..303: mol_type = genomic DNA
atgtatagtctggttccgacgcctacatttaagcgcgatctaaaacgactctccaagaagcattggccgatggacgaactaaagacggctgttaatctcctagctgctggtacaaatgctgaactattaagcaaaaagtatgcagatcatgccttgtcttcaagcagcgagtggaaaggatatcgtgaactacatgttgacggccctcgtggcgactggttgctaatctataaaattaagcagcaagatctcattttgaccctggttagaactggatctcatcataaccttttgggtaaatag

SEQ ID NO: 11 - DNA, 270 bp, Lactobacillus casei
  source 1..270: mol_type = genomic DNA
atggcagccacaaagaaagaaactcgcttgaatattcgtgttgatccggaattaaaaagtgctgctcaaatcgtagcaaatgatatgggcatcgacttgaccgcagctgttactatgttcatgacgaaaatggtgaaagatcacgccctcccgtttaccccaacaagtctaccagttgaaaccttacaggcgctgaaagaagcaaagcacccagagctgctcaaaaaatacagcacgcctgatgacatgtggagagacttgaatgtatag

SEQ ID NO: 12 - DNA, 210 bp, Lactobacillus acidophilus
  source 1..210: mol_type = genomic DNA
atgtgtttttttgttcaaaccaatcatacgagaaaggaactcctttttctaatgtttaaagaaataaatttaaggaatcattcatccttacacaaactattttacagtctcattcttcataaaccacatcctagaaaagaactattaccatatcaaatcaaacaaattcaagataaaatcaaagagatgaataaagatgaaaaaaactaa

SEQ ID NO: 13 - DNA, 342 bp, Lactobacillus acidophilus
  source 1..342: mol_type = genomic DNA
atgaaaaaaactaatcaatatatgaagtataagggttatgaaggttcaattgaatatactttggaggataagattctttttggtaaggttcaggggattaagagtcttatttcttacgaaggtaatacaatagatgaattggagaaggattttcaaggagccattgatgattatctaatgagctgtaaggaagatggagttataccagagaaaccttttaaaggtaattttaatgtgcgcattgatccttcattacatgaaaaattggctaattatgctgcaacgaagcaccaatcattgaatgctagtgtagaagaagctataaaaagatttttggcttag

SEQ ID NO: 14 - DNA, 228 bp, Lactobacillus plantarum
  source 1..228: mol_type = genomic DNA
ttgccaccaaccattcagcaattagcattgcgaaaattactaatgattgatcatgcggaaacaattaatgatttgagcctaccgcctgccaatcacttggaaaaattaagtcatgatcgccaagggcaatatagcatcaggattaataatcagtatcggatatgttttgcaattcgaaatggtaatgagttttatgatgttgaaatagtcgattatcatcatggttag

SEQ ID NO: 15 - DNA, 255 bp, Lactobacillus plantarum
  source 1..255: mol_type = genomic DNA
atgaatgaaatcccaacacctaaaattagtgaaattcttgaagaagagtttatggctcccttgcatatctctgcatattttttggctcaacagattggggtacccacgtcacggattcaagacttactacatgatcgtcgacaggtgacggttgatacatcgctgcggctaggacgattctttggagtgtcggatcgttatttcttggaacttcaaaatgatattgaaattcgtaatttgaaacagatacatggt

SEQ ID NO: 16 - DNA, 348 bp, Lactobacillus plantarum
  source 1..348: mol_type = genomic DNA
ttagaatggaaagtcgaagttgtaatacaccgtttgcgcgacgtgataaaaatcggcactgcgcatggtttcgatgtatgtgatactccgtcctgctctgggggtcgcatcaaacgaataaatttgggcagcagcaatttgtccgtgggtattgtaaccattaagactaaagtagccgggcaaatcacgcattgtagacgtaattggtgaaacaattacaaacccggtgttctgagtgtagagattactactaagaactacagctggccgacgtttctttatttcgcggccacgttgtgggtcaaaatcaatccagatgatgtctttttgtttaggtaaataagtcat

SEQ ID NO: 17 - DNA, 219 bp, Lactobacillus plantarum
  source 1..219: mol_type = genomic DNA
ttactcgacctcattccagtcgacatcggaccatgcttcagtcattggccgtttcggcttggagccggcataaggattctcgatacgtggtacgagtgtatatgactcaccatcacttgatgggatgagtaaccatcgttgtcctttcttaaaacgaccgtctttaggtagacttaatgccaatgaattaccacgactaactgtctttacctcaatcat

SEQ ID NO: 18 - DNA, 3535 bp, synthetic construct
  source 1..3535: mol_type = other DNA
  misc_feature 1..589: function = Origin of replication
  misc_feature 1778..2515: function = Erythromycin resistance
  misc_feature 2593..2621: function = pL promoter
  misc_feature 2635..2657: function = Ribosome-Binding Site (RBS)
  misc_feature 2667..3377: function = mcherry gene
  misc_feature 3384..3431: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggattatctctggcggtgttgacataaataccactggcggtgatactgagaacatcagcatttgtttaactttaagaaggagagaattcatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaagcaactagcataaccccttggggcctctaaacgggtcttgaggggttttttgctgagggagcagaatgtccgagactaattcatgaccaaaatcccttaacgtgagttttcgttccactgagcgtcagaccccgtagaaaagatcaaaggatcttc

SEQ ID NO: 19 - DNA, 3535 bp, synthetic construct
  source 1..3535: mol_type = other DNA
  misc_feature 1..589: function = origin of replication
  misc_feature 1778..2515: function = Erythromycin resistance
  misc_feature 2593..2620: function = pR promotor
  misc_feature 2635..2657: function = Ribosome-Binding Site (RBS)
  misc_feature 2667..3377: function = mcherry gene
  misc_feature 3384..3431: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggtctaacaccgtgcgtgttgactattttacctctggcggtgataatggttgaaaaattatttgtttaactttaagaaggagagaattcatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaagcaactagcataaccccttggggcctctaaacgggtcttgaggggttttttgctgagggagcagaatgtccgagactaattcatgaccaaaatcccttaacgtgagttttcgttccactgagcgtcagaccccgtagaaaagatcaaaggatcttc

SEQ ID NO: 20 - DNA, 6084 bp, synthetic construct
  source 1..6084: mol_type = other DNA
  misc_feature 1..589: function = origin of replication
  misc_feature 1778..2515: function = Erythromycin resistance
  misc_feature 5074..5125: function = pTlpA promoter
  misc_feature 5140..5162: function = Ribosome-Binding Site (RBS)
  misc_feature 5172..5882: function = mcherry gene
  misc_feature 5888..5935: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccaaccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattttaatttgtttgttagttagtttatttgttggtttgtttgtgttataatatcctctagaaataattttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaataactagcataaccccttggggcctctaaacgggtcttgaggggttttttgctgaaaggaggaactatatccgagtaaacttggtctgacagttaccaatgggagcagaatgtccgagactaattcatgaccaaaatcccttaacgtgagttttcgttccactgagcgtcagaccccgtagaaaagatcaaaggatcttc

SEQ ID NO: 21 - DNA, 7330 bp, synthetic construct
  source 1..7330: mol_type = other DNA
  misc_feature 1..589: function = origin of replication
  misc_feature 1778..2515: function = Erythromycin resistance
  misc_feature 5074..5125: function = pTlpA promoter
  misc_feature 5140..5162: function = Ribosome-Binding Site (RBS)
  misc_feature 5172..5882: function = mcherry gene
  misc_feature 5888..5935: function = T7 terminator
  misc_feature 5971..6010: function = P48
  misc_feature 6033..7148: function = Opti repressor Tlp
  misc_feature 7154..7181: function = T7Te terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccaaccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattttaatttgtttgttagttagtttatttgttggtttgtttgtgttataatatcctctagaaataattttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaataactagcataaccccttggggcctctaaacgggtcttgaggggttttttgctgaaaggaggaactatatccatatccgcaccgtaagttgttgacatggaacgaggaatgtgataatctgtgagtatagcggaggaggttgaccacgatgagaccagccacctatgaacctgagcagattattgaagccggtttagcccttcaggccgaaggtagaaacatcaccggtttcgctttaagaaaccaagtaggcggtggaaatcctactcgccttcgccaaatttgggatgaataccaggcaagtcagagtacagtcgttactgaacctgttgccgaacttcctgtagaggtcgctgaggaagtaaaggcagttagtgcagctctttcagagcgtatcacccagcttgcaaccgaacttaatgacaaagctgttcgtgcagctgagcgccgcgtagctgaagttaccagagctgctggtgagcaaacagctcaagctgagcgagagttggctgacgccgcacagaccgttgacgatttagaagaaaaacttgttgaattacaggaccgctatgattctcttactttagccttagagagcgaacgatcattacgccaacagcacgatgtagagatggcacaattaaaagagcgccttgccgctgccgaagaaaatacccgtcagagagaggagcgttaccaagagcaaaaaactgtcttacaggatgcattaaatgccgagcaagcccagcataaaaatacccgcgaggaccttcaaaaacgcttggaacagatttctgtcgaagcaaacgcccgaaccgaggaattgaaaagcgagagagacaaggttaatactttccttacccgacttgagagtcaagagaatgctcttgcaagcgagcgccagcagcatcttgcaactagagagactttgcagcagcgcttagaacaggctatcgcagacacccaggcacgtgcaggcgagatcgctttggagcgtgatagagttagctcattgacagcacgattagaatctcaggaaaaagcaagttctgagcagttagtccgcatgggtagcgaaattgcaagccttacagagcgatgcacacaattagagaaccaacgtgatgacgctcgcttagaaacaatgggtgagaaggagaccgtagcagctcttcgtggtgaagctgaggctttgaagagacagaaccagagtcttatggccgctttgtcaggaaataagcagacaggaggccaaaacgcctaaaataaggctcaccttcgggtgggcctttctgcgatcaggttatttgcataagctgagtaaacttggtctgacagttaccaatgggagcagaatgtccgagactaattcatgaccaaaatcccttaacgtgagttttcgttccactgagcgtcagaccccgtagaaaagatcaaaggatcttc

SEQ ID NO: 22 - DNA, 3535 bp, synthetic construct
  source 1..3535: mol_type = other DNA
  misc_feature 1..589: function = origin of replication
  misc_feature 1778..2515: function = Erythromycin resistance
  misc_feature 2581..2634: function = P23 promoter
  misc_feature 2635..2657: function = Ribosome-Binding Site (RBS)
  misc_feature 2667..3377: function = mcherry gene
  misc_feature 3384..3431: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggcatcctgatgacaaaaagagaaaattttgataaaatagtcttagaattaaattaaaaatttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaagcaactagcataaccccttggggcctctaaacgggtcttgaggggttttttgctgagggagcagaatgtccgagactaattcatgaccaaaatcccttaacgtgagttttcgttccactgagcgtcagaccccgtagaaaagatcaaaggatcttc

SEQ ID NO: 23 - DNA, 3533 bp, synthetic construct
  source 1..3533: mol_type = other DNA
  misc_feature 1..589: function = origin of replication
  misc_feature 1778..2515: function = Erythromycin resistance
  misc_feature 2586..2625: function = P48 promoter
  misc_feature 2633..2655: function = Ribosome-Binding Site (RBS)
  misc_feature 2665..3375: function = mcherry gene
  misc_feature 3383..3429: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggcatctcgtaagttgttgacatggaacgaggaatgtgataatctgtgagtatagcggtttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaagcaactagcataaccccttggggcctctaaacgggtcttgaggggttttttgctgagggagcagaatgtccgagactaattcatgaccaaaatcccttaacgtgagttttcgttccactgagcgtcagaccccgtagaaaagatcaaaggatcttc

SEQ ID NO: 24 - DNA, 6161 bp, synthetic construct
  source 1..6161: mol_type = other DNA
  misc_feature 1..589: function = origin of replication
  misc_feature 1778..2515: function = Erythromycin resistance
  misc_feature 2742..4088: function = Histidine protein kinase
  misc_feature 4166..4836: function = Response regulator transcription factor
  misc_feature 5133..5180: function = SPP promoter
  misc_feature 5248..5270: function = Ribosome-Binding Site (RBS)
  misc_feature 5280..5990: function = mcherry gene
  misc_feature 5998..6044: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggggtcgacttcaatagagttcttaacgttaatccgaaaaaaactaacgttaatattaaaaaataagatccgcttgtgaattatgtataatttgattagactaaagaataggagaaagtatgatgatatttaaaaaactttctcgttaagataggttgttggtgagcatgttatatacggatgtatcggtttccttaatgcaaaattttgttgctatcttattaatttttctattatatagatatattcaaagaaagataacatttaaacggatcatattagatattttaatagcgattattttttcaatattatatctgtttatttcagatgcgtcattacttgtaatggtattaatgcgattagggtggcattttcatcaacaaaaagaaaataagataaaaacgactgatacagctaatttaattctaattatcgtgatccagttattgttagttgcggttgggactattattagtcagtttaccatatcgattatcaaaagtgatttcagccaaaatatattgaacaatagtgcaacagatataactttattaggtattttctttgctgttttatttgacggcttgttctttatattattgaagaataagcggactgaattacaacatttaaatcaagaaatcattgaattttcgttagaaaaacaatattttatatttatatttattttatttatagtaatagaaattattttagcagttgggaatcttcaaggagtaacagccacgatattattaaccattatcattattttttgtgtccttatcgggatgactttttggcaagtgatgctttttttgaaggcttattcgattcgccaagaagccaatgaccaattggtccggaatcaacaacttcaagattatctagtcaatatcgaacagcagtacaccgaattacggcgatttaagcatgattatcaaaacatcttattatcgttggagagttttgccgaaaagggcgatcagcaacagtttaaggcgtattaccaagaattattagcacaacggccaattcaaagtgaaatccaaggggcagtcattgcacaactcgactacttgaaaaatgatcctattcgaggattagtcattcaaaagtttttggcagccaaacaggctggtgttactttaaaattcgaaatgaccgaaccaatcgaattagcaaccgctaatctattaacggttattcggattatcggtattttattagacaatgcgattgaacaagccgttcaagaaaccgatcaattggtgagttgtgctttcttacaatctgatggtttaatcgaaattacgattgaaaatacggccagtcaagttaagaatctccaagcattttcagagttaggctattcaacgaaaggcgctggtcgggggactggtttagctaatgtgcaggatttgattgccaaacaaaccaatttattcttagaaacacagattgaaaatagaaagttacgacagacattgatgattacggaggaaacttaatttgtatcccgtttatttattagaggatgatttacagcaacaagcgatttatcagcaaattatcgcgaatacgattatgattaacgaatttgcaatgactttaacatgcgctgccagtgatactgagacattgttggcggcaattaaggatcagcaacgaggtttattctttttggatatggaaattgaggataaccgccaagccggtttagaagtggcaactaagattcggcagatgatgccgtttgcgcaaattgtcttcattacaacccacgaggaactgacattattaacgttagaacgaaaaatagcgcctttagattacattctcaaggaccaaacaatggctgaaatcaaaaggcaattgattgatgatctattgttagctgagaagcaaaacgaggcggcagcgtatcaccgagaaaatttatttagttataaaataggtcctcgctttttctcattaccattaaaggaagttgtttatttatatactgaaaaagaaaatccgggtcatattaatttgttagccgttaccagaaaggttacttttccaggaaatttaaatgcgctggaagcccaatatccaatgctctttcggtgtgataaaagttacttagttaacctatctaatattgccaattatgacagtaaaacacggagtttaaaatttgtagatggcagtgaggcaaaagtctcgttccggaaatcacgggaactagtggccaaattaaaacaaatgatgtagcgcctgcagcacgccaaatgatcccagtaaaaagccacccgcatggcgggtggctttttattagccctagaagggcttcccacacgcatttcagcgccttagtgccttagtttgtgaatcataggtggtatagtcccgaaatacccgtctaaggaattgtcagataggcctaatgactggcttttataatatgagataatgccgactgtactttttacagtcggttttctaatgtcactaacctgccccgttagttgaagaaggtttttatattacagctccagatctaccggtgggcccatattaacgtttaaccgataaagttgaacgttaatattttttttgcgcagaaatggtaaattgaagcataatagtcttgtaaggtatttagctggctggcgtaaagtatgctttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaagcaactagcataaccccttggggcctctaaacgggtcttgaggggttttttgctgaaaggaggaactatatgagcagaatgtccgagactaattcatgaccaaaatcccttaacgtgagttttcgttccactgagcgtcagaccccgtagaaaagatcaaaggatcttc

SEQ ID NO: 25 - DNA, 3535 bp, synthetic construct
  source 1..3535: mol_type = other DNA
  misc_feature 1..589: function = origin of replication
  misc_feature 1778..2515: function = Erythromycin resistance
  misc_feature 2581..2622: function = tuf promoter
  misc_feature 2635..2657: function = Ribosome-Binding Site (RBS)
  misc_feature 2667..3377: function = mcherry gene
  misc_feature 3384..3431: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggcatctctgtttacaaatcagattaggctatatataatatttaaggattctcagtgatgtttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaagcaactagcataaccccttggggcctctaaacgggtcttgaggggttttttgctgagggagcagaatgtccgagactaattcatgaccaaaatcccttaacgtgagttttcgttccactgagcgtcagaccccgtagaaaagatcaaaggatcttc

SEQ ID NO: 26 - DNA, 9432 bp, synthetic construct
  source 1..9432: mol_type = other DNA
  misc_feature 1..589: function = origin of replication
  misc_feature 1778..2515: function = Erythromycin resistance
  misc_feature 5074..5125: function = pTlpA promoter
  misc_feature 5140..5162: function = Ribosome-Binding Site (RBS)
  misc_feature 5172..5882: function = mcherry gene
  misc_feature 5888..5935: function = T7 terminator
  misc_feature 8058..8315: function = Txe
  misc_feature 8308..8577: function = Axe
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccaaccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattttaatttgtttgttagttagtttatttgttggtttgtttgtgttataatatcctctagaaataattttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaataactagcataaccccttggggcctctaaacgggtcttgaggggttttttgctgaaaggaggaactatatccgaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctttgtcgatcctaccatccactcgacacacccgccagcggccgctgccaagcttccgagctcgatacttctttcaaattcaagttaaatttatattccattacttctcctccaatatataaaaattacttaactgtatccaaatttaatatgtaaataaatcattaatttttagctaaattctatgagaagtttatttttgttgtttttgctaattgactaaaactagtacaaaaaatttaaaatagattattaaacctattgcttattctattcagtcatatatataagcgtcatcctttagtaactaacatcccacactacaatagtattataacatatctattcaacttcaaattgttgaaatttaagccatttatatttacttttcctcaactttagaaatgattttactaaccttatatccgtaatacgcgtaacaaacacattcattaataacaatcaacactaagctatattagctttttaaatgactagggtaaataaaataaggatcaaaaacttttaagtttctgaccctttccttacttccgattggttaatagtgatcttttgcagaataaataaatatcgtttcattttcaactctatatatcagtctatgttcatctgtaattcttctggaccattttccagataaatcatgctttaatggctcaggttttcctaatccagcaaagggggaacgatcgatatcttttattaacttgttaatcttttttatattgcttttgtttccttgctcatgccaataaagataatcatcccaagcatcatcagaccaagccttaatcatcagattcaacctcgattaagtcatgtgttttaaatgcacctttggagaattgttcatctcctcgacgaattttttccatgacgtaattattagaaagtgttctcaacgtttcttgcatagaatcataatctctttttgataatacaacaactgtatcttctacatctttacttgttacaataagtgtttcagcatcctcattaacttgtttcatataactacgtaaattttggcggaaatttgaataagctactgcttccattcctttcaccccttatttatttctataaaacaattgtacattatattgtacaattaagcaaatgattaatattctaacttcttataattaaaagtgctatcacttttagtttttaggataaaaggtgacgtcactttttgagtgctggtacctcaaatcctgctttttcttgggcaatcaataaatctaacgcaatacacataatcctggattcacttttgccgtacttctgctttagttgctgcaattctttttgtctcttttcatccaacgtaacgggtaaccgtttatttcctattgcaattggcattagtcgcttatggatcctgaacttgaccgtaaacgaatgccatatcaacgatttgagaggaaaaaaggcaataatcatttataccgaaaagtgagaatgtgtgaaataaggacaaaaaacctagtaactaagggaatggttcttatttcgcaccaatcgctccaaccgcccccgccgcttaggtggtgcgaaataagcgggcaacacccgataacctaagagaaagaaggagatccatatgaataaaatcattcctctattgtcagtcctatttgttggcgaaatacttatgcgccatgctgcagctgtgcatttaaacttcgtagtatttttggcaatactggtattttgtttggtcttttggcgctttgtattagcccttgtgagtctactattcaagatgattgcagccatattgagtaaacttggtctgacagttaccaatgggagcagaatgtccgagactaattcatgaccaaaatcccttaacgtgagttttcgttccactgagcgtcagaccccgtagaaaagatcaaaggatcttc

SEQ ID NO: 27 - DNA, 5208 bp, synthetic construct
  source 1..5208: mol_type = other DNA
  misc_feature 1..589: function = origin of replication
  misc_feature 1778..2515: function = Erythromycin resistance
  misc_feature 3279..3548: function = DinJ antitoxin
  misc_feature 3541..3843: function = YafQ toxin
  misc_feature 4198..4249: function = pTlpA promoter
  misc_feature 4264..4286: function = Ribosome-Binding Site (RBS)
  misc_feature 4296..5006: function = mcherry gene
  misc_feature 5012..5059: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccttggtatgatggcagttacgcttccatttgtgcgaagaaaaagccagaaatccggttgaattcatctcacatgatgttatcccactgtgtttacattgggataattcgtgatataattaggttgttagataggaaggagtggttagcaatggcagccacaaagaaagaaactcgcttgaatattcgtgttgatccggaattaaaaagtgctgctcaaatcgtagcaaatgatatgggcatcgacttgaccgcagctgttactatgttcatgacgaaaatggtgaaagatcacgccctcccgtttaccccaacaagtctaccagttgaaaccttacaggcgctgaaagaagcaaagcacccagagctgctcaaaaaatacagcacgcctgatgacatgtggagagacttgaatgtatagtctggttccgacgcctacatttaagcgcgatctaaaacgactctccaagaagcattggccgatggacgaactaaagacggctgttaatctcctagctgctggtacaaatgctgaactattaagcaaaaagtatgcagatcatgccttgtcttcaagcagcgagtggaaaggatatcgtgaactacatgttgacggccctcgtggcgactggttgctaatctataaaattaagcagcaagatctcattttgaccctggttagaactggatctcatcataaccttttgggtaaatagaactacgaaaggccgtccaaaaaaggcggcttttgtgttgtggtcaaccagcagattcattcgtcaatatacacgtgacttaacgccaatcttcaatcagagattagaaaccgcgaactcgtcggcccagtaaaaatgttcagctatcgagatgagttgggtgatctggggaagtgagcatcaataagatgccaatctttatcatgcagggttggaacgaaaatttaacccaatgttccaaatgcgtggtgatgtcggcgatataggcgccagcaaccgcacctgtggcgccggtgatgccggccacgatgcgtccggcgtagaggatcgagatcgatctcgatcccgcgaaattttaatttgtttgttagttagtttatttgttggtttgtttgtgttataatatcctctagaaataattttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaataactagcataaccccttggggcctctaaacgggtcttgaggggttttttgctgaaaggaggaactatatccgagtaaacttggtctgacagttaccaatgggagcagaatgtccgagactaattcatgaccaaaatcccttaacgtgagttttcgttccactgagcgtcagaccccgtagaaaagatcaaaggatcttc

SEQ ID NO: 28 - DNA, 5196 bp, synthetic construct
  source 1..5196: mol_type = other DNA
  misc_feature 1..589: function = origin of replication
  misc_feature 1778..2515: function = Erythromycin resistance
  misc_feature 3328..3555: function = HigA antitoxin
  misc_feature 3567..3821: function = HigB toxin
  misc_feature 4186..4237: function = pTlpA promoter
  misc_feature 4252..4274: function = Ribosome-Binding Site (RBS)
  misc_feature 4284..4994: function = mcherry gene
  misc_feature 5000..5047: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccactctggttggttcctagctaaaataggggtcgtactagtcgggtaatcagatgaattggatatttgggtgtggtcagctaataataggagcaagagtcggatggattgagatatacacgtgatacgggtataatcgattaagaattttgcagataaggaaactcacaaggtttaccaacaaaaattctctaaacaattgccaccaaccattcagcaattagcattgcgaaaattactaatgattgatcatgcggaaacaattaatgatttgagcctaccgcctgccaatcacttggaaaaattaagtcatgatcgccaagggcaatatagcatcaggattaataatcagtatcggatatgttttgcaattcgaaatggtaatgagttttatgatgttgaaatagtcgattatcatcatggttaggagaccaaaaaatgaatgaaatcccaacacctaaaattagtgaaattcttgaagaagagtttatggctcccttgcatatctctgcatattttttggctcaacagattggggtacccacgtcacggattcaagacttactacatgatcgtcgacaggtgacggttgatacatcgctgcggctaggacgattctttggagtgtcggatcgttatttcttggaacttcaaaatgatattgaaattcgtaatttgaaacagatacatggtgctgaatatgcacagataaaaaagtatcaagtcagttaaataaggacgtagaccgtagttgcttggtagcgtactacggtttttgcaaatataacctaaactgataagcagcagatcttaaattttaaggttatcaacaggtggataaagcagtggcgtttgtattatccacctgttgataatcattgagttggttaattgtactttcactactgggttaactggtcgtgagaaaaatggtcaagcattagtttcgtggtgatgtcggcgatataggcgccagcaaccgcacctgtggcgccggtgatgccggccacgatgcgtccggcgtagaggatcgagatcgatctcgatcccgcgaaattttaatttgtttgttagttagtttatttgttggtttgtttgtgttataatatcctctagaaataattttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaataactagcataaccccttggggcctctaaacgggtcttgaggggttttttgctgaaaggaggaactatatccgagtaaacttggtctgacagttaccaatgggagcagaatgtccgagactaattcatgaccaaaatcccttaacgtgagttttcgttccactgagcgtcagaccccgtagaaaagatcaaaggatcttc

SEQ ID NO: 29 - DNA, 5182 bp, synthetic construct
  source 1..5182: mol_type = other DNA
  misc_feature 1..589: function = origin of replication
  misc_feature 1778..2515: function = Erythromycin resistance
  misc_feature 3284..3631: function = MazF toxin
  misc_feature 3631..3849: function = MazE antitoxin
  misc_feature 4172..4223: function = pTlpA promoter
  misc_feature 4238..4260: function = Ribosome-Binding Site (RBS)
  misc_feature 4270..4980: function = mcherry gene
  misc_feature 4986..5033: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccgaaggatttacggtcaacgagtgtctttcggacacgtccgttgatattgttatttgactcgtggtcttactccaatcaattatactgacaagcatcgcaaatataaaaccgccctacctaacctgagtaagttacagggcgaagtagtgctgattagaatggaaagtcgaagttgtaatacaccgtttgcgcgacgtgataaaaatcggcactgcgcatggtttcgatgtatgtgatactccgtcctgctctgggggtcgcatcaaacgaataaatttgggcagcagcaatttgtccgtgggtattgtaaccattaagactaaagtagccgggcaaatcacgcattgtagacgtaattggtgaaacaattacaaacccggtgttctgagtgtagagattactactaagaactacagctggccgacgtttctttatttcgcggccacgttgtgggtcaaaatcaatccagatgatgtctttttgtttaggtaaataagtcattactcgacctcattccagtcgacatcggaccatgcttcagtcattggccgtttcggcttggagccggcataaggattctcgatacgtggtacgagtgtatatgactcaccatcacttgatgggatgagtaaccatcgttgtcctttcttaaaacgaccgtctttaggtagacttaatgccaatgaattaccacgactaactgtctttacctcaatcatatcaaggcctcctcgtattaataaacacactatttatgcaacgagtataacacgtattacgaaataagacagctaaagcaatcgcgataactaattgataagcacacggtttttagtaattcatcaataaaggaagagactaattaatgtcgccgtaactcagtgttatcaagttggatggcccgctttagtattttccacctttagcctcttgtggtgatgtcggcgatataggcgccagcaaccgcacctgtggcgccggtgatgccggccacgatgcgtccggcgtagaggatcgagatcgatctcgatcccgcgaaattttaatttgtttgttagttagtttatttgttggtttgtttgtgttataatatcctctagaaataattttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaataactagcataaccccttggggcctctaaacgggtcttgaggggttttttgctgaaaggaggaactatatccgagtaaacttggtctgacagttaccaatgggagcagaatgtccgagactaattcatgaccaaaatcccttaacgtgagttttcgttccactgagcgtcagaccccgtagaaaagatcaaaggatcttc

SEQ ID NO: 30 - DNA, 4992 bp, synthetic construct
  source 1..4992: mol_type = other DNA
  misc_feature 1..589: function = origin of replication
  misc_feature 1778..2515: function = Erythromycin resistance
  misc_feature 3239..3448: function = HicA toxin
  misc_feature 3435..3776: function = HicB antitoxin
  misc_feature 3982..4033: function = pTlpA promoter
  misc_feature 4048..4070: function = Ribosome-Binding Site (RBS)
  misc_feature 4080..4790: function = mcherry gene
  misc_feature 4796..4843: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgccccagcctgttgtagctctttgcgcaataaatcatttattttgtctggattgaatagagcttgagcaaaatctttggtaaaatcattcatgaaggagttcttctttctgtatgtgtttttttgttcaaaccaatcatacgagaaaggaactcctttttctaatgtttaaagaaataaatttaaggaatcattcatccttacacaaactattttacagtctcattcttcataaaccacatcctagaaaagaactattaccatatcaaatcaaacaaattcaagataaaatcaaagagatgaataaagatgaaaaaaactaatcaatatatgaagtataagggttatgaaggttcaattgaatatactttggaggataagattctttttggtaaggttcaggggattaagagtcttatttcttacgaaggtaatacaatagatgaattggagaaggattttcaaggagccattgatgattatctaatgagctgtaaggaagatggagttataccagagaaaccttttaaaggtaattttaatgtgcgcattgatccttcattacatgaaaaattggctaattatgctgcaacgaagcaccaatcattgaatgctagtgtagaagaagctataaaaagatttttggcttaggtgaaagacgcttggaatttttcaagcgtttttttcatgaaaacaattgctaaattcattgaatgagtgtactattttagtataacaatagttgaacgggtgatgtcggcgatataggcgccagcaaccgcacctgtggcgccggtgatgccggccacgatgcgtccggcgtagaggatcgagatcgatctcgatcccgcgaaattttaatttgtttgttagttagtttatttgttggtttgtttgtgttataatatcctctagaaataattttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaataactagcataaccccttggggcctctaaacgggtcttgaggggttttttgctgaaaggaggaactatatccgagtaaacttggtctgacagttaccaatgggagcagaatgtccgagactaattcatgaccaaaatcccttaacgtgagttttcgttccactgagcgtcagaccccgtagaaaagatcaaaggatcttc

SEQ ID NO: 31 - DNA, 5643 bp, synthetic construct
  source 1..5643: mol_type = other DNA
  misc_feature 1..589: function = origin of replication
  misc_feature 1778..2515: function = Erythromycin resistance
  misc_feature 2747..3094: function = MazF toxin
  misc_feature 3094..3312: function = MazE antitoxin
  misc_feature 3714..3983: function = DinJ antitoxin
  misc_feature 3976..4278: function = YafQ toxin
  misc_feature 4633..4684: function = pTlpA promoter
  misc_feature 4699..4721: function = Ribosome-Binding Site (RBS)
  misc_feature 4731..5441: function = mcherry gene
  misc_feature 5447..5494: function = T7 terminator
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggagcgaggaagcggaagagaaggatttacggtcaacgagtgtctttcggacacgtccgttgatattgttatttgactcgtggtcttactccaatcaattatactgacaagcatcgcaaatataaaaccgccctacctaacctgagtaagttacagggcgaagtagtgctgattagaatggaaagtcgaagttgtaatacaccgtttgcgcgacgtgataaaaatcggcactgcgcatggtttcgatgtatgtgatactccgtcctgctctgggggtcgcatcaaacgaataaatttgggcagcagcaatttgtccgtgggtattgtaaccattaagactaaagtagccgggcaaatcacgcattgtagacgtaattggtgaaacaattacaaacccggtgttctgagtgtagagattactactaagaactacagctggccgacgtttctttatttcgcggccacgttgtgggtcaaaatcaatccagatgatgtctttttgtttaggtaaataagtcattactcgacctcattccagtcgacatcggaccatgcttcagtcattggccgtttcggcttggagccggcataaggattctcgatacgtggtacgagtgtatatgactcaccatcacttgatgggatgagtaaccatcgttgtcctttcttaaaacgaccgtctttaggtagacttaatgccaatgaattaccacgactaactgtctttacctcaatcatatcaaggcctcctcgtattaataaacacactatttatgcaacgagtataacacgtattacgaaataagacagctaaagcaatcgcgataactaattgataagcacacggtttttagtaattcatcaataaaggaagagactaattaatgtcgccgtaactcagtgttatcaagttggatggcccgctttagtattttccacctttagcctcttgtgatgctcacgatacgggttactgatgatgaacatgcccttggtatgatggcagttacgcttccatttgtgcgaagaaaaagccagaaatccggttgaattcatctcacatgatgttatcccactgtgtttacattgggataattcgtgatataattaggttgttagataggaaggagtggttagcaatggcagccacaaagaaagaaactcgcttgaatattcgtgttgatccggaattaaaaagtgctgctcaaatcgtagcaaatgatatgggcatcgacttgaccgcagctgttactatgttcatgacgaaaatggtgaaagatcacgccctcccgtttaccccaacaagtctaccagttgaaaccttacaggcgctgaaagaagcaaagcacccagagctgctcaaaaaatacagcacgcctgatgacatgtggagagacttgaatgtatagtctggttccgacgcctacatttaagcgcgatctaaaacgactctccaagaagcattggccgatggacgaactaaagacggctgttaatctcctagctgctggtacaaatgctgaactattaagcaaaaagtatgcagatcatgccttgtcttcaagcagcgagtggaaaggatatcgtgaactacatgttgacggccctcgtggcgactggttgctaatctataaaattaagcagcaagatctcattttgaccctggttagaactggatctcatcataaccttttgggtaaatagaactacgaaaggccgtccaaaaaaggcggcttttgtgttgtggtcaaccagcagattcattcgtcaatatacacgtgacttaacgccaatcttcaatcagagattagaaaccgcgaactcgtcggcccagtaaaaatgttcagctatcgagatgagttgggtgatctggggaagtgagcatcaataagatgccaatctttatcatgcagggttggaacgaaaatttaacccaatgttccaaatgcgtggtgatgtcggcgatataggcgccagcaaccgcacctgtggcgccggtgatgccggccacgatgcgtccggcgtagaggatcgagatcgatctcgatcccgcgaaattttaatttgtttgttagttagtttatttgttggtttgtttgtgttataatatcctctagaaataattttgtttaactttaagaaggagatataccatgatggtttcaaagggtgaagaagataacatggctatcatcaaggaattcatgcgtttcaaggttcacatggaaggttcagttaacggtcacgaattcgaaatcgaaggtgaaggtgaaggtcgtccatacgaaggtactcaaactgctaagttaaaggttactaagggtggtccattaccattcgcttgggatatcttatcaccacaattcatgtacggttcaaaggcttacgttaagcacccagctgatatcccagattacttaaagttatcattcccagaaggtttcaagtgggaacgtgttatgaacttcgaagatggtggtgttgttactgttactcaagattcatcattacaagatggtgaattcatctacaaggttaagttacgtggtactaacttcccatcagatggtccagttatgcaaaagaagactatgggttgggaagcttcatcagaacgtatgtacccagaagatggtgctttaaagggtgaaatcaagcaacgtttaaagttaaaggatggtggtcactacgatgctgaagttaagactacttacaaggctaagaagccagttcaattaccaggtgcttacaacgttaacatcaagttagatatcacttcacacaacgaagattacactatcgttgaacaatacgaacgtgctgaaggtcgtcactcaactggtggtatggatgaattatacaagtaaaataactagcataaccccttggggcctctaaacgggtcttgaggggttttttgctgaaaggaggaactatatccgagtaaacttggtctgacagttaccaatgggagcagaatgtccgagactaattcatgaccaaaatcccttaacgtgagttttcgttccactgagcgtcagaccccgtagaaaagatcaaaggatcttc

SEQ ID NO: 32 - DNA, 2675 bp, synthetic construct
  source 1..2675: mol_type = other DNA
  misc_feature 1..589: function = origin of replication
  misc_feature 1778..2515: function = Erythromycin resistance
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgctgcgagtcagtgagcgagaaggattattcggctggttgagacgttaaaatgataaaggttgtattaatcttatattacggttataatgtactcaacttaataaatgaacgcaaaaaaaagaaccctcaacttagcagagttaggattcacgacttatcagcacaacctgataagattttcgatagcaagtactaccaatacaagctatctaacttggtactattataacatgtaggctaagtttttcaaccattgatacttaaagtaaacggttgttatcgggaatcttaacagaaacctgatagcaaccgtttttttgttattcaatggttagcaaccatcaaagcaactaaaggctggaaacctgttcttagctagtaaaacctcccgtgagtgtcgttcgtgaccccgcttgcagttaacaacataggtatgctaaaccttgtcgagatcaacgcgactaaagacgtggctggaagactaggaaatgatacggacaggctaactattaacgcagattattcgggttgctgctaaaaccaactctaataatagttagtgcaagggctggttgagcttaaattgtctgataaagagttctctctttatactgcaaaagaagcgcagttattcacgattaggataactgtttgagagagcctaagggcttgacccttgatggtttaagcaccgctatgcgtgcgggatccccttagaagcaaacttagagtgtgttgatagtgcattatcttaaaattttgtataataggaattgaagttaaattagatgctaaaaataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttcaggagagattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggaatattccttacaatttaagcacacaaattattaaaaaagtggtttttgaaagccgtgcgtctgacatctatctgactgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatataagtactttgtttcaaaatgggtcaatcgagaatatcgtcaactgtttactaaaaatcagtttcgtcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagaccggggagcagaatgtccgagactaattcatgaccaaaatcccttaacgtgagttttcgttccactgagcgtcagaccccgtagaaaagatcaaaggatcttc

SEQ ID NO: 33 - protein, 19 aa, synthetic construct
MAGNSSNFIHKIKQIFTHR

(19) United States
(12) Patent Application Publication
(10) Pub. No.: US 2026/0071226 A1
(43) Pub. Date: Mar. 12, 2026

(54) Novel Genetic Tools

(71) Applicant: INM - Leibniz-Institut für Neue Materialien gemeinnützige GmbH, Saarbrücken (DE)

(72) Inventors: Shrikrishnan Sankaran, Saarbrücken (DE); Sourik Dey, Saarbrücken (DE); Marc Blanch Asensio, Saarbrücken (DE)

(21) Appl. No.: 18/994,669

(22) PCT Filed: Jul. 7, 2023
(86) PCT No.: PCT/EP2023/068901

§ 371 Date: Jan. 15, 2025

(30) Foreign Application Priority Data
Jul. 28, 2022 (DE) 10 2022 119 024.2

(51) Int. Cl. C12N 15/74; C07K 14/315; C07K 14/335

(52) U.S. Cl. CPC C12N 15/746; C07K 14/315; C07K 14/335

(57) ABSTRACT

An expression cassette for expression in Lactobacillus includes a promoter operatively linked to a nucleic acid encoding a protein to be expressed, the promoter being substantially identical to the P_{tlpA} promoter.

### FIELD OF THE INVENTION

The invention relates to novel genetic tools for Lactobacillus. It relates to in particular a promoter for expression of proteins, in particular a vector comprising such a promoter. The vector may also additionally comprise at least one toxin-antitoxin system.

### Prior Art

Lactobacilli (Lactobacillus) are gram-positive, rod-shaped lactic acid bacteria (LAB) which typically occur as commensals in humans and animals. Their phenotypic stress-resistance properties allow them to colonize a broad range of microenvironments in the host, such as intestine, skin, vagina, nasal cavity and pharynx. They frequently offer health benefits such as anti-inflammatory, antipathogenic and immunomodulatory activity. That is why they are one of the largest classes of probiotics, and a number of species are used clinically to treat a multitude of diseases such as ulcerative colitis, mastitis, atopic dermatitis, bacterial vaginosis and periodontitis. In addition to their health benefits, Lactobacilli are also essential for numerous fermentation processes in the food industry, for example in the production of yogurt, cheese, sourdough bread, beer and wine. Because of this ubiquity in daily life, there is considerable interest in genetically improving and expanding the capabilities of these bacteria for both medical and industrial purposes. In the medical field, Lactobacilli are being developed as live biotherapeutic products (LBPs) which produce and deliver drugs directly at the site of diseases such as ulcerative colitis. To study the pharmacokinetics of these therapeutic bacteria and their colonization in the body, said bacteria have been manipulated to express reporter proteins which can be imaged in situ. In industry, Lactobacilli are considered to be alternative hosts to recombinant expression by E. coli because they have two crucial advantages: (i) they do not produce endotoxins, and the status of many strains is “Generally Recognized as Safe” (GRAS), which minimizes the risk of purified therapeutic proteins causing toxic effects in humans, and (ii) the infrastructure for culture thereof is already well established and optimized in the food industry.

Despite this potential, the key constraints on Lactobacillus engineering are the lack of well-characterized genetic tools and the insufficient understanding of biochemical pathways. More than two decades of careful testing and screening of phylogenetically related bacteria have produced a handful of reliable tools for use in Lactobacillus, such as constitutive and inducible promoters, operators, replicons, retention modules, signal peptides, etc. Most of these tools were developed in a small number of species found to be suitable for genetic modification, one of the most commonly used species being Lactiplantibacillus plantarum (or Lactobacillus plantarum). Although the genomic integration of genes was demonstrated in these bacteria, it was with the aid of plasmids that the greatest variety of functions was achieved. However, the number of well-characterized genetic tools available are still tiny compared to the E. coli toolbox and must be increased in order to design the type of cycles required for applications in the medical or food industry.

### Object

It is an object of the invention to provide a way of expressing proteins in Lactobacillus at a high yield.

### Achievement

This object is achieved by the inventions having the features of the independent claims. Advantageous developments of the inventions are characterized in the dependent claims. The wording of all the claims is hereby incorporated in this description by reference. The inventions also include all meaningful and in particular all stated combinations of independent and/or dependent claims.

For the sake of clarity, specific terms used in the description are defined and described as follows:

“Associated with/operatively linked” refers to two nucleic acids which are physically or functionally linked to one another. For example, a promoter or a regulatory DNA sequence is referred to as “associated with” a DNA sequence encoding RNA or a protein when the two sequences are operatively linked to one another or are arranged such that the regulatory DNA sequence influences the expression level of the coding or structural DNA sequence.

A “coding sequence” is a nucleic acid sequence which is transcribed into RNA such as mRNA, rRNA, tRNA, snRNA, sense RNA or antisense RNA. Preferably, the RNA is then translated in an organism in order to produce a protein.

“Expression cassette” refers to a nucleic acid sequence which is capable of controlling the expression of a specific nucleotide sequence in a suitable host cell and which includes a promoter functionally linked to the nucleotide sequence of interest functionally linked to termination signals. In addition, it generally includes sequences required for proper translation of the nucleotide sequence. In the expression cassette containing the nucleotide sequence of interest, at least one of its components may be heterologous compared to at least one of its other components. The expression cassette may also be a naturally occurring cassette which, however, has been obtained in a recombinant form suitable for heterologous expression. Generally, however, the expression cassette is heterologous in relation to the host, i.e., the specific nucleic acid sequence of the expression cassette does not occur naturally in the host cell and must have been introduced into the host cell or an ancestor of the host cell by a transformation event. The nucleotide sequence in the expression cassette may be expressed under the control of a constitutive promoter or an inducible promoter which initiates transcription only when the host cell has been exposed to a specific external stimulus.

In addition, the expression cassette preferably comprises at least one ribosome binding site. It is preferably arranged between the promoter and the protein to be expressed, i.e., start codon (ATG) of the protein to be expressed, in the 5′-3′ direction.

An expression cassette containing a nucleotide sequence of interest may be chimeric, i.e., at least one of its components is heterologous in relation to at least one of its other components. An expression cassette may also contain a native promoter which drives the native gene, but was obtained in a recombinant form suitable for heterologous expression. Such use of an expression cassette means that it does not occur naturally in the cell into which it was introduced.

An expression cassette may also optionally contain a transcriptional and/or translational termination region (i.e., termination region) which is functional in Lactobacillus. Available for use in expression cassettes are a multitude of transcription terminators, which are responsible for the termination of transcription after the heterologous nucleotide sequence of interest and for correct mRNA polyadenylation. The termination region may be native to the transcription initiation region, it may be native to the operatively linked nucleotide sequence of interest, it may be native to Lactobacillus, or it may come from some other source (i.e., foreign or heterologous to the promoter, the nucleotide sequence of interest, Lactobacillus, or any combination thereof). Furthermore, the native transcription terminator of a coding sequence may also be used. Any available terminator known to function in Lactobacillus may be used in the context of the present invention.

The term “expression”, when used in relation to a polynucleotide, such as a gene, an ORF or a portion thereof, or a transgene in Lactobacillus, refers to the process of converting the genetic information encoded in a gene into RNA (e.g., mRNA, rRNA, tRNA or snRNA) by “transcription” of the gene (i.e., by the enzymatic action of an RNA polymerase) and, if applicable, into protein by “translation” of the mRNA (e.g., if a gene encodes a protein). Gene expression may be regulated in many phases of the process. In the case of antisense or dsRNA constructs, expression may for example only refer to the transcription of antisense RNA or dsRNA. In some embodiments, the term “expression” refers to the transcription and stable accumulation of sense RNA (mRNA) or functional RNA. The term “expression” may also refer to the production of proteins.

A “gene” is a defined region within a genome that comprises a coding nucleic acid sequence and that typically also contains other nucleic acids, especially regulatory nucleic acids responsible for controlling the expression, i.e., the transcription and translation, of the coding part. A gene may also contain other sequences: 5′ and 3′ untranslated sequences and termination sequences. Further elements which may be present are, for example, introns. The regulatory nucleic acid sequence of the gene is normally not operatively linked to the associated nucleic acid sequence occurring in nature and would therefore be a chimeric gene.

A “heterologous” nucleic acid sequence or “heterologous” nucleic acid molecule is a nucleic acid sequence or nucleic acid molecule that is not naturally associated with the host cell into which it is introduced. A heterologous nucleic acid sequence or heterologous nucleic acid molecule may comprise a chimeric sequence, for example a chimeric expression cassette, in which the promoter and the coding region originate from multiple parent organisms. The promoter sequence may be a constitutive promoter sequence, a chemically inducible promoter sequence, a temperature-inducible promoter sequence or a stress-inducible promoter sequence. Heterologous is also referred to as endogenous.

A “homologous” nucleic acid sequence is a nucleic acid sequence which is naturally associated with a host cell into which it is introduced.

“Homologous recombination” is the reciprocal exchange of nucleic acid fragments between homologous nucleic acid molecules.

“Identity” or “percentage identity” refers to the degree of similarity between two nucleic acid or protein sequences. In sequence comparison, one sequence generally serves as the reference sequence by means of which the test sequences are compared. When using a sequence comparison algorithm, test and reference sequences are input into a computer, with or without specification of partial sequence coordinates, and program parameters for the sequence algorithm are defined. The sequence comparison algorithm then calculates the percentage sequence identity for the test sequence(s) relative to the reference sequence on the basis of the specified program parameters. The expression “substantially identical”, in connection with two nucleic acids or two amino acid sequences, refers to two or more sequences or partial sequences having at least about 50% nucleotide or amino acid residue identity when they are compared and aligned for maximum matching, as measured by one of the following sequence comparison algorithms or by visual inspection. In certain embodiments, substantially identical sequences have at least about 60% or at least about 70% or at least about 80% or at least about 85% or even at least about 90% or 95% nucleotide or amino acid residue identity. In certain embodiments, substantial identity exists over a region of the sequences that is at least about 50 residues long, or over a region of at least about 100 residues, or the sequences are substantially identical over at least about 150 residues. In further embodiments, the sequences are substantially identical when they are identical over the entire length of the coding regions. The foregoing also applies to regulatory sequences, such as promoters, based in this case on their nucleotide sequence.

An optimal alignment of the sequences to be compared can be achieved, for example, using the local homology algorithm from Smith & Waterman, Adv. Appl. Math. 2:482 (1981), by the homology alignment algorithm from Needleman & Wunsch, J. Mol. Biol. 48:443 (1970), by the similarity search method from Pearson & Lipman, Proc. Natl. Acad. Sci. USA 85:2444 (1988), by computer-assisted implementations of these algorithms (GAP, BESTFIT, FASTA and TFASTA in the Wisconsin Genetics Software Package, Genetics Computer Group, 575 Science Dr., Madison, WI) or by visual inspection.

An example of an algorithm suitable for determining percentage sequence identity and sequence similarity is the BLAST algorithm, which is mentioned in Altschul et al., J. Mol. Biol. 215:403-410 (1990). The software for carrying out BLAST analyses is publicly accessible through the National Center for Biotechnology Information (http://www.ncbi.nlm.nih.gov/). This algorithm first ascertains high-scoring segment pairs (HSPs) by identifying short words of length W in the query sequence that either match a word of the same length in a database sequence or exceed a positive-scoring threshold T when they are aligned with said word. T is referred to as the neighborhood word score threshold (Altschul et al., 1990). These initial neighborhood word hits serve as the starting point to search for longer HSPs containing these words. The word hits are then stretched in both directions along each sequence as far as the cumulative alignment score can be increased. The cumulative results are calculated for nucleotide sequences with the parameters M (reward value for a pair of matching residues; always >0) and N (penalty value for nonmatching residues; always <0). For amino acid sequences, a scoring matrix is used for calculating cumulative scoring. The stretching of the word hits in each direction is stopped when the cumulative alignment score deviates from its maximum achieved value by the amount X, when the cumulative score is zero or lower owing to the accumulation of one or more negative-scoring residue alignments or when the end of one of the two sequences is reached. The parameters W, T and X of the BLAST algorithm determine the sensitivity and speed of alignment. The default values used by the BLASTN program (for nucleotide sequences) are a word length (W) of 11, an expectation (E) of 10, a cutoff of 100, M=5, N=−4, and a comparison of both strands. For amino acid sequences, the BLASTP program uses as default a word length (W) of 3, an expectation (E) of 10 and the BLOSUM62 scoring matrix (see Henikoff & Henikoff, Proc. Natl. Acad. Sci. USA 89:10915 (1989)).

Besides calculating percentage sequence identity, the BLAST algorithm also carries out a statistical analysis of the similarity between two sequences (see for example Karlin & Altschul, Proc. Natl. Acad. Sci. USA 90:5873-5787 (1993)). One measure of similarity provided by the BLAST algorithm is the smallest sum probability (P(N)), which indicates the probability with which a match between two nucleotide or amino acid sequences would occur randomly. For example, a test nucleic acid sequence is regarded as similar to a reference sequence if the smallest sum probability in a comparison of the test nucleic acid sequence with the reference nucleic acid sequence is less than about 0.1, preferably less than about 0.01 and particularly preferably less than about 0.001.

Another indication that two nucleic acids are substantially identical is the fact that the two molecules hybridize to one another under stringent conditions. The expression “specifically hybridize with” refers to the binding, duplex formation or hybridization of a molecule with a specific nucleotide sequence only under stringent conditions when said sequence is present in a complex mixture of (e.g., total cellular) DNA or RNA. “Binding substantially” refers to the complementary hybridization between a probe nucleic acid and a target nucleic acid and includes minor mispairings which can be compensated for by reducing the stringency of the hybridization media in order to achieve the desired detection of the target nucleic acid sequence.

“Stringent hybridization conditions” and “stringent hybridization wash conditions” in connection with nucleic acid hybridization experiments such as Southern hybridization and Northern hybridization are sequence-dependent and differ in various environmental parameters. Longer sequences hybridize specifically at higher temperatures. A comprehensive guide on the hybridization of nucleic acids can be found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. In general, the hybridization and wash conditions chosen are highly stringent and they are about 5° C. below the thermal melting point (Tm) for the specific sequence at a specific ionic strength and a specific pH. Typically, a probe hybridizes with its target sequence, but not with other sequences, under “stringent conditions”.

The Tm value is the temperature (at a defined ionic strength and a defined pH) at which 50% of the target sequence hybridizes with a perfectly matched probe. Highly stringent conditions are chosen such that they are line with the Tm value for a specific probe. An example of stringent hybridization conditions for the hybridization of complementary nucleic acids having more than 100 complementary residues on a filter in a Southern or Northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a wash with 0.2×SSC at 65° C. for 15 minutes (see J. Sambrook, et al., Molecular Cloning: A Laboratory Manual, 3d Ed., Cold Spring Harbor, NY: Cold Spring Harbor Laboratory Press (2001), for a description of the SSC buffer). A high-stringency wash procedure is frequently preceded by a low-stringency wash procedure in order to remove the background probe signal. An example of a wash procedure of medium stringency for a duplex of, for example, more than 100 nucleotides is 1×SSC at 45° C. for 15 minutes. An example of a wash procedure of low stringency for a duplex of, for example, more than 100 nucleotides is 4-6×SSC at 40° C. for 15 minutes. For short probes (e.g., about 10 to 50 nucleotides), stringent conditions typically include salt concentrations of less than about 1.0 M Na ions, typically a concentration of about 0.01 to 1.0 M Na ions (or other salts) at pH 7.0 to 8.3, and the temperature is typically at least about 30° C. Stringent conditions may also be achieved by the addition of destabilizing agents such as formamide. In general, a signal-to-noise ratio of 2× (or higher) compared to a nonrelated probe in a hybridization assay points to the detection of specific hybridization. Nucleic acids which do not hybridize with one another under stringent conditions are nevertheless substantially identical when the proteins for which they code are essentially identical. This is the case, for example, when a copy of a nucleic acid is made by using the maximum codon degeneracy allowed by the genetic code.

Listed below are examples of hybridization/wash conditions which can be used for the cloning of homologous nucleotide sequences substantially identical to the reference nucleotide sequences of the present invention: A reference nucleotide sequence preferably hybridizes with the reference nucleotide sequence in 7% sodium dodecyl sulfate (SDS), 0.5 M NaPO₄, 1 mM EDTA at 50° C. with washes in 2×SSC, 0.1% SDS at 50° C., preferably in 7% sodium dodecyl sulfate (SDS), 0.5 M NaPO₄, 1 mM EDTA at 50° C. with washes in 1×SSC, 0.1% SDS at 50° C., especially in 7% sodium dodecyl sulfate (SDS), 0.5 M NaPO₄, 1 mM EDTA at 50° C. with washes in 0.5×SSC, 0.1% SDS at 50° C., more preferably in 7% sodium dodecyl sulfate (SDS), 0.5 M NaPO₄, 1 mM EDTA at 50° C. with washes in in 0.1×SSC, 0.1% SDS at 50° C., yet more preferably in 7% sodium dodecyl sulfate (SDS), 0.5 M NaPO₄, 1 mM EDTA at 50° C. with washes in 0.1×SSC, 0.1% SDS at 65° C.

Another indication that two nucleic acids or proteins are substantially identical is that the protein encoded by the first nucleic acid is immunologically cross-reactive with the protein encoded by the second nucleic acid or binds specifically thereto. For instance, a protein is typically substantially identical to a second protein when the two proteins differ only by conservative substitutions.

A nucleic acid sequence and a reference nucleic acid sequence are “isocoding” when the nucleic acid sequence encodes a polypeptide having the same amino acid sequence as the polypeptide encoded by the reference nucleic acid sequence.

An “isolated” nucleic acid molecule or isolated toxin is a nucleic acid molecule or toxin which exists outside of its natural environment as a result of human activity and is therefore not a product of nature. An isolated nucleic acid molecule or toxin may be in purified form or may exist in a non-natural environment, for example in a recombinant microbial cell.

A “nucleic acid molecule” or “nucleic acid sequence” is a segment of a single- or double-stranded DNA or RNA that can be isolated from any source. In connection with the present invention, the nucleic acid molecule is typically a DNA segment. In some embodiments, the nucleic acid molecules according to the invention are isolated nucleic acid molecules.

The terms “protein”, “peptide” and “polypeptide” are used interchangeably herein.

The term “codon-optimized” sequence refers to the nucleotide sequence of a recombinant, transgenic or synthetic polynucleotide in which the codons are chosen to reflect the particular codon bias a host cell may have. This is done in a way that preserves the amino acid sequence of the polypeptide encoded by the codon-optimized polynucleotide. In certain embodiments, the nucleotide sequence of the recombinant DNA construct contains a sequence which has been codon-optimized for the cell (e.g., an animal cell, plant cell or fungal cell) in which the construct is to be expressed.

A “promoter” is a nontranslated DNA sequence upstream of the coding region that contains the binding site for the RNA polymerase and initiates the transcription of the DNA. The promoter region may also contain other elements which act as regulators of gene expression.

“Regulatory elements” refers to sequences involved in controlling the expression of a nucleotide sequence. Regulatory elements include a promoter functionally linked to the nucleotide sequence of interest and, optionally, termination signals. They generally also include sequences required for proper translation of the nucleotide sequence.

“Transformation” is a method for introducing a heterologous nucleic acid into a host cell or an organism. In particular, “transformation” means the stable integration of a DNA molecule into the genome (nucleus or plastid) of an organism of interest.

“Transformed/transgenic/recombinant” refers to a host organism such as a bacterium or plant into which a heterologous nucleic acid molecule has been introduced. The nucleic acid molecule may be stably integrated into the genome of the host organism or may be present as an extrachromosomal molecule. Such an extrachromosomal molecule may be self-replicating. Transformed cells, tissues or plants are to be understood to mean not only the end products of a transformation process, but also the transgenic progeny thereof. A “nontransformed”, “nontransgenic” or “nonrecombinant” host refers to a wild-type organism, for example a bacterium, which the heterologous nucleic acid molecule does not contain.

Nucleotides are indicated by their bases using the following standard abbreviations: adenine (A), cytosine (C), thymine (T) and guanine (G). Amino acids are likewise indicated using the following standard abbreviations: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y) and valine (Val; V).

The object is achieved, inter alia, by an expression cassette for expression in Lactobacillus comprising a promoter operatively linked to a nucleic acid encoding at least one protein, the promoter being substantially identical to the P_{tlpA} promoter, preferably to a sequence substantially identical to sequence ID No. 1. Preferably, the promoter is at least 90%, at least 91%, at least 92%, at least 93%, at least 94%, at least 95%, at least 96%, at least 97%, at least 98%, at least 99% or 100% identical to the sequence of SEQ ID No. 1.

The promoter is a heterologous promoter, preferably from Salmonella typhimurium.

It has been found that, surprisingly, this promoter allows very high expression of the operatively linked protein in Lactobacillus. Expression is increased especially within the range of temperatures between 31° C. and 45° C., especially within a temperature range of 37° C. to 43° C.

It has been found that, surprisingly, the Pₜₗₚₐ promoter allows very strong expression, especially in the preferred temperature range, even when its associated repressor is absent.

In a preferred embodiment, the expression cassette includes at least one ribosome binding site which is operatively linked to the promoter and which is preferably arranged between the promoter and the encoded protein. In a preferred embodiment, the distance between the ribosome binding site and the start codon of the encoded protein is at least 8 base pairs, in particular at least 9 base pairs, preferably from 9 to 24 base pairs, in particular 9 to 15 base pairs.

The invention also relates to a vector, in particular a plasmid, comprising the expression cassette.

The vector may be any vector suitable for Lactobacillus.

The vectors may additionally include other regulatory elements.

Lactobacillus in the context of the application is preferably understood to mean a bacterium of the order lactic acid bacteria (Lactobacillales), preferably of the family Lactobacilluseae, particularly preferably of the genus Lactobacillus, Lacticaseibacillus, Lactiplantibacillus.

Preference is given to the species Lactobacillus reuteri, Lactobacillus paracasei (Lacticaseibacillus paracasei), Lactobacillus plantarum (Lactiplantibacillus plantarum), Lactobacillus johnsonii, Lactobacillus rhamnosus, Lactobacillus acidophilus, Lactobacillus salivarius, Lactobacillus casei (Lacticaseibacillus casei), Lactococcus lactis, preferably Lactobacillus plantarum (Lactiplantipacillus plantarum), particularly preferably Lactobacillus plantarum WCFS1 (Lactiplantibacillus plantarum WCFS1).

The protein encoded by the nucleic acid may be a homologous or heterologous protein in relation to Lactobacillus, preferably a heterologous protein.

The protein may be adapted according to the use of Lactobacillus.

The invention also relates to a Lactobacillus comprising the at least one expression cassette.

The invention also relates to a Lactobacillus comprising at least one expression cassette according to the invention.

The invention also relates to a Lactobacillus comprising at least one vector comprising the at least one expression cassette.

In one embodiment of the invention, the expression cassette is operatively linked to at least one toxin-antitoxin system. This means that, in addition to the expression cassette, at least one toxin and at least one antitoxin can be expressed. Such systems are known from antibiotic-free genetic stabilization. When a protein is expressed, a toxin and an associated antitoxin are also expressed. The toxin generally has a growth-inhibiting effect on the Lactobacillus. The antitoxin neutralizes the toxin, which is degraded more slowly than the antitoxin. When cell division yields a cell without the antitoxin gene, said cell is killed by the toxin. These systems are therefore preferably used for gene maintenance, in particular plasmid maintenance, in cells over multiple generations. Since they do not require antibiotics for this purpose, they are suitable for use in living systems, too, for example the gastrointestinal tract.

The at least one toxin-antitoxin system may be homologous or heterologous.

In a preferred embodiment, the at least one toxin-antitoxin system is arranged on the same vector as the expression cassette.

In one embodiment of the invention, the expression cassette is operatively linked to at least two toxin-antitoxin systems, preferably at least two different toxin-antitoxin systems. In the case of a vector, these elements are arranged on the vector.

In one embodiment of the invention, the toxin-antitoxin system is linked to a promoter, preferably to a promoter other than the promoter of the expression cassette. What is important is only that the promoter is at least also active when the promoter of the expression cassette is activated. Preferably, the promoter is a constitutive promoter which is not operated by an external stimulus. This plasmid retention is thus always active when the plasmid is in the cell. In this case, “operatively linked” can also mean that the expression cassette and the TA systems are arranged on the same vector and the operative link is the plasmid retention due to the toxin-antitoxin system.

Many toxin-antitoxin systems are known.

Such systems are differentiated into different types (Singh G. et al. Current Research in Microbial Sciences 2, 2021, 100047, Bacterial toxin-antitoxin modules: classification, functions, and association with persistence).

In the case of type I TA systems, the antitoxin is an antisense RNA which inhibits the translation of the mRNA of the toxins. An example thereof is the hok-sok module (Gerdes, K., Bech, F. W., Jørgensen, S. T., Løbner-Olesen, A., Rasmussen, P. B., Atlung, T., Boe, L., Karlstrom, O., Molin, S., von Meyenburg, K., 1986. Mechanism of postsegregational killing by the hok gene product of the parB system of plasmid R1 and its homology with the relF gene product of the E. coli relB operon. EMBO J 5 (8), 2023-2029.). In the case of type II TA systems, the toxin and the antitoxin are proteins which form a complex with one another. Many of the toxins are endoribonucleases or inhibitors of DNA gyrase.

In the case of a type III TA system, the antitoxin is an RNA which directly interacts with the toxin protein.

In the case of a type IV TA system, the antitoxin and the toxin are proteins which do not directly interact with one another, but bind competitively to a protein.

In the case of a type V TA system, the antitoxin is an endoribonuclease against the mRNA of the toxin.

In the case of a type VI TA system, the toxin inhibits replication, whereas the antitoxin causes decomposition of the toxin.

In the case of a type VII TA system, the antitoxin modifies the toxin.

The TA system may be a type I, type II, type III, type IV, type V or type VI system.

Examples of type I TA systems are Hok and Sok, Fst and RNAII, TisB and IstR, LdrB and RdlD, FlmA and FlmB, lbs and Sib, TxpA/BmT and RatA, SymE and SymR, and XXCV2162 and ptaRNAl.

Examples of type II TA systems are CcdB and CcdA, ParE and ParD, MaxF and MazE, yafO and yafN, HicA and HicB, Kid and Kis, Zeta and Epsilon, DarT and DarG, Txe and Axe, YafQ and DinJ, HigB and HigA, HipA and HipB, PhD and Doc, RelB and RelE, VapB and VapC, RnlA and RnlB, and MqsR and MqsA.

An example of a type III TA system is ToxN and Toxl.

An example of a type V TA system is GoT and GoS.

An example of a type VI TA system is SocA and SocB.

In a preferred embodiment, the at least one toxin-antitoxin system is a type II TA system, preferably selected from the group comprising Txe (SEQ ID No. 8)/Axe (SEQ ID No. 9), YafQ (SEQ ID No. 10)/DinJ (SEQ ID No. 11), HicA (SEQ ID No. 12)/HicB (SEQ ID No. 13), HigB (SEQ ID No. 14)/HigA (SEQ ID No. 15) and MazF (SEQ ID No. 16)/MazE (SEQ ID No. 17). The sequences are preferably adjacent to one another, but they may also overlap.

Such a toxin-antitoxin system can increase the retention of a plasmid over multiple generations of Lactobacillus. It has now been found that, surprisingly, the presence of at least two toxin-antitoxin systems, in particular two toxin-antitoxin systems, most particularly two different toxin-antitoxin systems, distinctly improves retention.

Preference is given to at least 15%, in particular at least 20% retention, after 100 generations.

When at least two toxin-antitoxin systems are present, preference is given to a retention of at least 40%, preferably over 50%, after 100 generations.

In a preferred embodiment, the at least one toxin-antitoxin system achieves a G₅₀ value of at least 50, in particular at least 60. The G₅₀ value is the number of generations until the proportion of plasmid-containing cells falls below 50%. In the case of at least two toxin-antitoxin systems, preference is given to a G₅₀ value of over 100.

These retention values are lower than in the case of retention under the influence of antibiotics. However, these systems function without external addition of antibiotics. In addition, the loss of plasmid leads to the formation of a natural organism without any genetic modification. A Lactobacillus comprising such a vector can therefore also be referred to as a temporary genetically modified organism (GMO). In the course of generations, this organism loses the genetic modification. This is especially of interest if the Lactobacillus is to be used in a medical or human environment.

The vector may additionally contain other regulatory elements, for example antibiotic resistances, which facilitate production.

The invention also relates to the use of the promoter of the expression cassette according to the invention in Lactobacillus.

The invention also relates to the use of the vector according to the invention in Lactobacillus, in particular in Lactobacillus plantarum.

Especially the improved expression of protein due to the promoter according to the invention allows many new applications. For instance, the high level of expression also allows the detection of expressed fluorescent proteins in vivo, which was hardly possible with the known promoters owing to the low level of expression. This allows better use of the promoter for analysis and diagnosis.

### Materials and Methods

Further details and features will become apparent from the following description of preferred exemplary embodiments in connection with the dependent claims. Here, the particular features may be realized alone or a plurality thereof may be realized in combination with one another. The ways of achieving the object are not limited to the exemplary embodiments.

The exemplary embodiments are shown schematically in the figures. Identical reference numerals in the individual figures refer to identical or functionally identical elements or mutually corresponding elements in terms of their functions. Specifically, the figures show:

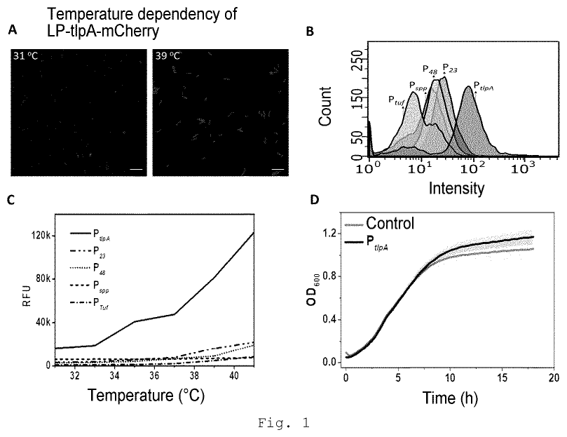

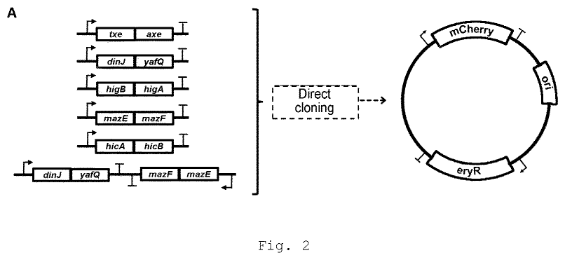

FIG. 1 A) Fluorescence microscopy of P_{tlpA}-driven mCherry expression in L. plantarum WCFS1, cultured at 31° C. and 39° C. for 18 h. Scale bar=10 μm. B) Flow cytometry analysis of P_{tlpA}, P₂₃, P₄₈, Pₛₚₚ and P_{Tuf} promoter-controlled mCherry expression in L. plantarum WCFS1 after 18 hours of incubation at 37° C. C) Fluorescence spectroscopy analysis of P_{tlpA}, P₂₃, P₄₈, Pₛₚₚ and P_{Tuf} promoter-controlled mCherry expression after 18 hours of incubation at temperatures from 31° C. to 41° C. D) Measurement of the growth rate (OD₆₀₀) of the control (empty vector) and P_{tlpA}-driven mCherry expression in L. plantarum WCFS1 for 18 hours at 37° C. In C and D, the lines correspond to the mean of three biological replicates and the weak bands correspond to the standard deviation;

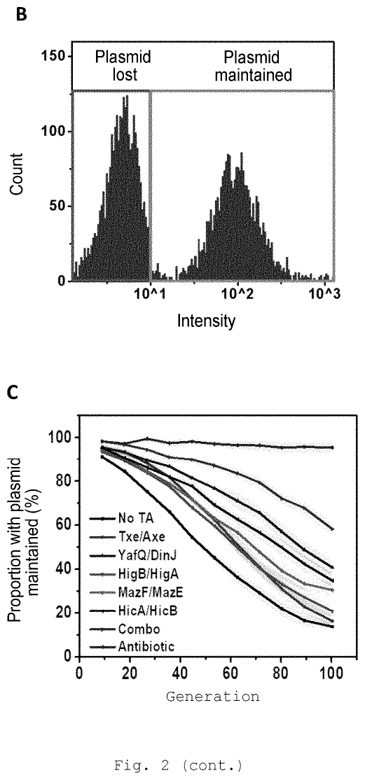

FIG. 2A) Schematic illustration of the direct cloning of the various toxin-antitoxin gene modules into the plasmid with Pₜₗₚₐ-controlled mCherry expression. B) Flow cytometry histogram of the strain with a plasmid which does not contain a TA module after 50 generations of serial passage in the absence of antibiotics. The right box corresponds to the bacterial population containing the plasmid and the left box corresponds to the bacterial population without the plasmid in the absence of selection pressure. C) Graphical representation of the extent of plasmid retention due to the TA modules in L. plantarum WCFS1 for 100 generations compared to the absence of TA system (No TA) or antibiotic control (Antibiotic). The lines correspond to the mean of three biological replicates, and the weak bands are the standard deviation; the order of the measurements from top to bottom is: Antibiotic, Combo, YafQ/DinJ; HicA/HicB, MazF/MazE, HigB/HigA, Txe/Axe, No TA);

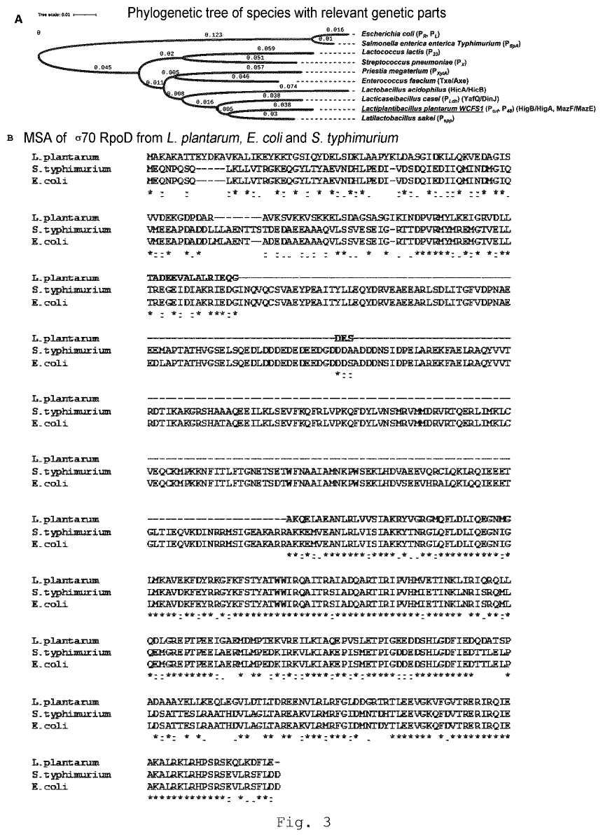

FIG. 3 A) Phylogenetic tree showing the distances between the species from which various genetic parts were tested in L. plantarum. The blue markings correspond to the bacteria from which promoters were adapted in this study, and the orange markings correspond to the bacteria from which TA systems were adapted. L. plantarum WCFS1 is marked green because both promoters and TA systems from this bacterium were adapted. B) Homology analysis of σ70 RpoD genes from L. plantarum, E. coli and S. typhimurium. The height and brightness of the yellow bars indicate the extent to which individual residues are conserved across all 3 bacteria;

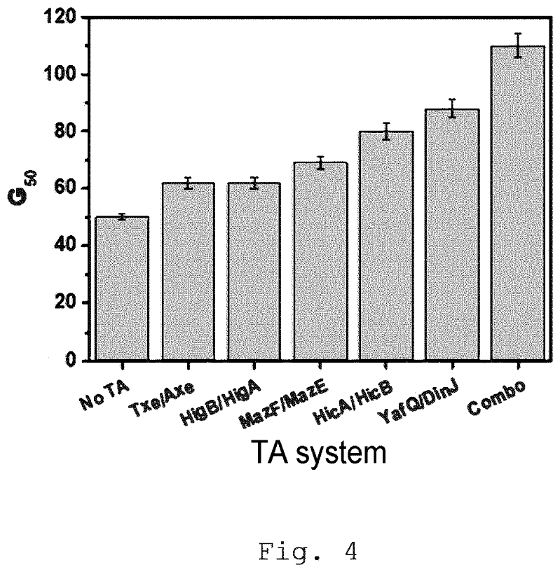

FIG. 4 G50 values of the various TA systems (No TA: no TA system, Combo: 2 TA systems); and

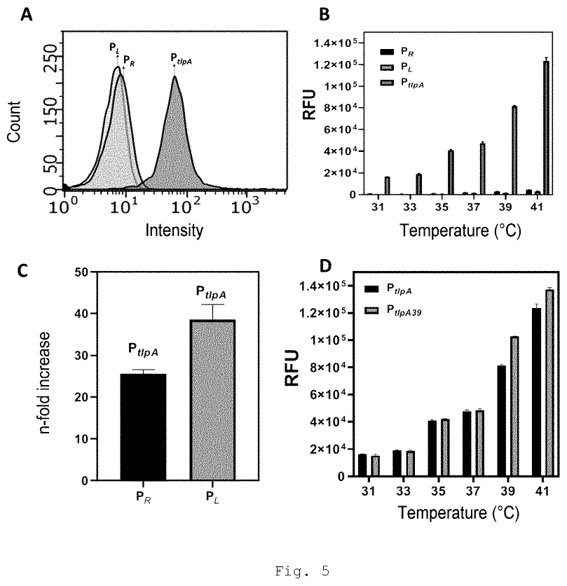

FIG. 5A) Flow cytometry analysis of P_{R}, P_{L} and P_{TlpA} promotor-encoded mcherry constructs in L. plantarum WCFS1 after 18 h of incubation at 37° C. B) Fluorescence spectroscopy analysis of P_{R}, P_{L} and P_{TlpA} promotor-encoded mcherry constructs after 18 hours of incubation at a temperature gradient from 31° C. to 41° C. The data are based on three biological replicates. C) Fold change (n-fold increase) in P_{TlpA} promotor-based mcherry expression compared to the P_{R} and P_{L} promotors at 37° C. D) RFU plot of P_{TlpA} and P_{TlpA39}-based mcherry expression after 18 hours of incubation at a temperature gradient from 31° C. to 41° C. The data correspond to three biological replicates carried out independently;

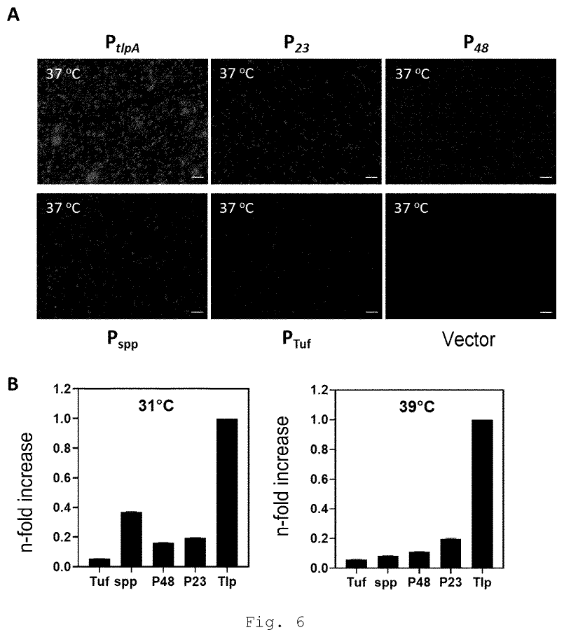

FIG. 6A) Fluorescence microscopy images of P_{TlpA}, P₂₃, P₄₈, Pₛₚₚ/P_{Tuf}-encoded mCherry constructs and empty vector constructs in L. plantarum WCFS1, cultured at 37° C. for 18 h (scale=10 μm). B) Fold change in P_{TlpA}, P₂₃, P₄₈ and Pₛₚₚ-encoded mCherry constructs in relation to P_{Tuf} promoter-based mcherry expression at 31° C. and 39° C. The data are based on three biological replicates carried out independently;

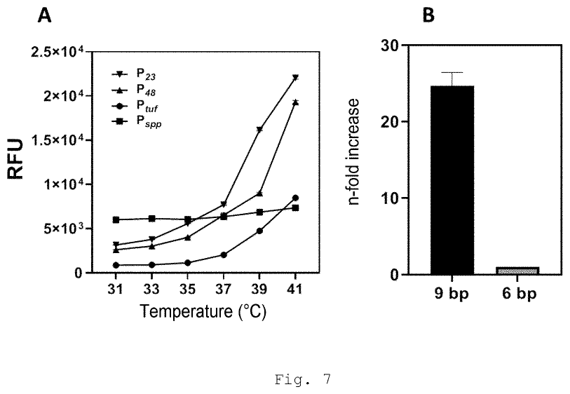

FIG. 7A) RFU plot of P₂₃, P₄₈ and P_{Tuf}-encoded mcherry constructs exhibiting increased fluorescence expression compared to Pₛₚₚ-based protein expression within the temperature gradient from 31° C. to 41° C. The data are based on three biological replicates carried out independently. B) Fold change in P_{TlpA} promoter-based mcherry expression with a 9 bp spacer (between the RBS and the start codon) compared to the P_{TlpA}-6 bp spacer construct;

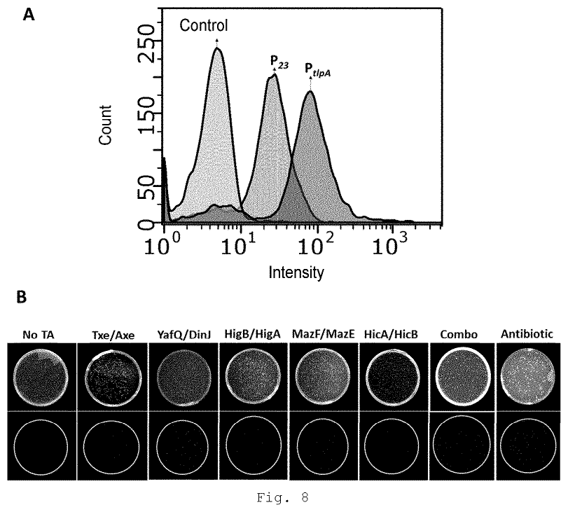

FIG. 8 A) Flow cytometry plots showing that strains with P₄₈-driven mCherry expression produce relatively low intensities, with part of the population overlapping with the signal coming from bacteria which do not express fluorescent proteins (control). In comparison, the signal of P_{tlpA} is clearly distinct from that of the control. B) Agar plate-based analysis of plasmid retention provided by the various TA systems. The total number of colonies can be determined from the bright-field images (top row), and the colonies visible in the fluorescence images (bottom row) are the plasmid-containing bacteria;

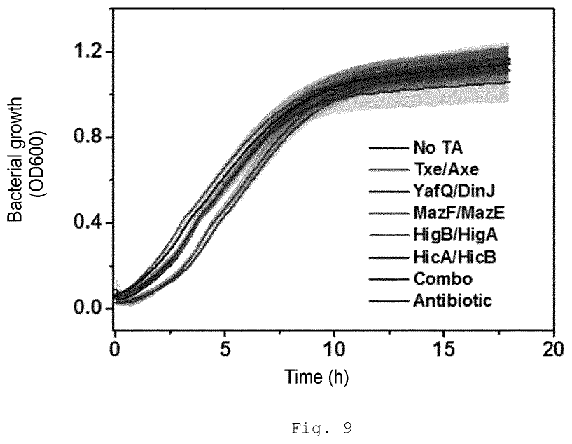

FIG. 9 Measurement of the growth rate (OD₆₀₀) Of P_{tlpA}-mcherry constructs in combination with the TA modules compared to the P_{tlpA}-mcherry construct grown in the absence (without TA) and in the presence (antibiotic) of selection pressure for 18 hours at 37° C. The data are based on three biological replicates and the weak bands are the respective standard deviations (No TA: no TA system; Combo: 2 TA systems; Antibiotic: antibiotic present);

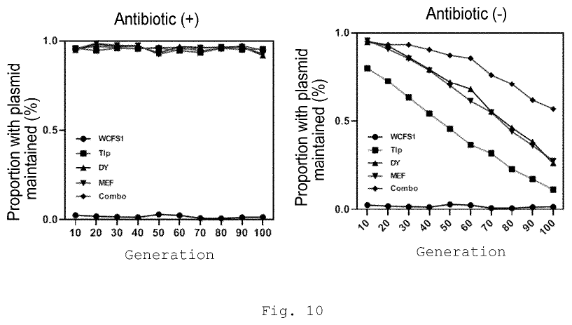

FIG. 10 Maintenance of different plasmids under antibiotic control (A) and under toxin-antitoxin control (B) (Combo: 2 TA systems);

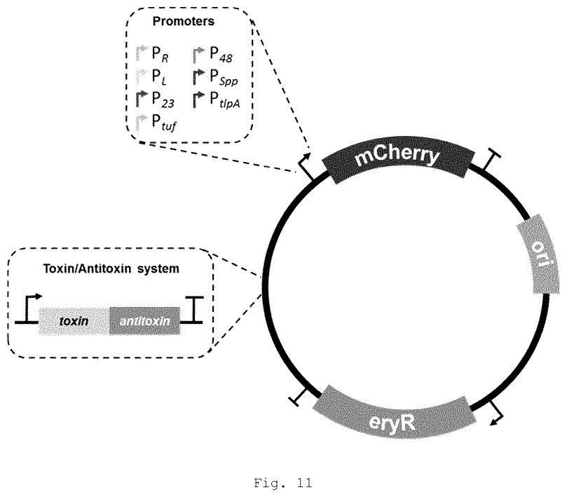

FIG. 11 Schematic illustration of the vector with origin of replication (ori); eryR: erythromycin resistance; toxin-antitoxin; the arrows represent promoters and T represents terminators;

### STRAINS, MEDIA AND PLASMIDS

The parent strain used for characterizing promoter strength and plasmid retention was L. plantarum WCFS1 (ATCC number BAA-793). The strain was kept in the media developed by De Man, Rogosa and Sharpe (MRS). The culture media, antibiotics and supplementary reagents were purchased from Carl Roth GmbH, Germany. The growth medium was supplemented with 10 μg/ml erythromycin for culturing of the manipulated L. plantarum WCFS1 strains. The plasmids pSIP403 and pLp_3050sNuc used in this study were provided by Prof. Lars Axelsson (Addgene Plasmid #122028) (Sørvig E, Mathiesen G, Naterstad K, Eijsink V G, Axelsson L. High-level, inducible gene expression in Lactobacillus sakei and Lactobacillus plantarum using versatile expression vectors. Microbiology. 2005; 151(7):2439-49) and Prof. Geir Mathiesen (Addgene Plasmid No. 122030) (Mathiesen G, Sveen A, Brurberg M B, Fredriksen L, Axelsson L, Eijsink V G. Genome-wide analysis of signal peptide functionality in Lactobacillus plantarum WCFS1. BMC genomics. 2009; 10(1):1-13) and the plasmid pTlpA39-Wasabi was provided by Prof. Mikhail Shapiro (Addgene Plasmid #86116) (Piraner D I, Abedi M H, Moser B A, Lee-Gosselin A, Shapiro M G. Tunable thermal bioswitches for in vivo control of microbial therapeutics. Nature chemical biology. 2017; 13(1):75-80). The plasmid pUC-GFP-AT was provided by Prof. Chris Barnes (Addgene Plasmid No. 133306) (Fedorec A J, Ozdemir T, Doshi A, Ho Y-K, Rosa L, Rutter J, et al. Two new plasmid post-segregational killing mechanisms for the implementation of synthetic gene networks in Escherichia coli. Iscience. 2019; 14:323-34). The L. plantarum WCFS1 strain was provided by Prof. Gregor Fuhrmann (Helmholtz Institute for Pharmaceutical Research Saarland). The sequence-verified genetic constructs made in this study were preserved in an E. coli DH5α strain.

### Molecular Biology

The genetic constructs developed in this study are based on the pLp3050sNuc/pSIP403 vector backbone. The HiFi Assembly Master Mix, the Quick Blunting Kit and the T4 DNA Ligase enzyme were purchased from New England BioLabs (NEB, Germany). PCR was carried out using the Q5 High Fidelity 2× Master Mix (NEB) and primers from Integrated DNA Technologies (IDT) (Louvain, USA). The oligonucleotide gene fragments were purchased as eBlocks from IDT (Coralville, USA). They were optimized for maximum expression in the host strain using the Java Codon Optimization Tool (JCAT) or the IDT Codon Optimization Tool (Coralville, USA). For plasma extraction and DNA cleanup, kits from Qiagen GmbH (Hilden, Germany) and Promega GmbH (Walldorf, Germany) were used. Sanger sequencing of colony PCR-amplified genetic segments was carried out by Eurofins Genomics GmbH (Ebersberg, Germany), with selection of the option of an additional DNA purification step. The promoter sequences used in this study are listed in Table 1.

L. plantarum WCFS1—Preparation of Competent Cells and DNA Transformation

A single colony of L. plantarum WCFS1 was inoculated in 5 mL of MRS medium and cultured overnight at 37° C. with shaking (250 rpm). The primary culture was diluted in a ratio of 1:50 (v/v) in a 25 mL secondary culture consisting of MRS media and 1% (w/v) glycine premixed in a ratio of 4:1. The secondary culture was incubated at 37° C. and 250 rpm until an OD600 of 0.8 was reached. The cells were then pelleted by centrifugation at 4000 rpm (3363×g) for 10 minutes at 4° C. The pellet was washed twice with 5 mL of ice-cold 10 mM MgCl₂ and then twice with 5 mL and 1 mL of ice-cold Sac/Gly solution [10% (v/v) glycerol (Gly) and 1 M sucrose (Sac) in a ratio of 1:1 (v/v)]. Lastly, the residual supernatant was discarded and the pellet was resuspended in 500 μl of Sac/Gly solution. The competent cells were then divided into 60 μl aliquots for DNA transformation. For all transformations, 1 μg of dsDNA (circular plasmid or DNA-ligated mixture) was added to the competent cells and they were then transferred to cooled electroporation cuvettes of 2 mm distance (Bio-Rad Laboratories GmbH, Germany). The transformation by electroporation was carried out with a single 1.8 kV pulse, immediately followed by addition of 1 ml of lukewarm MRS medium. The mixture was incubated for a recovery phase of 3 hours at 37° C. and 250 rpm. The recovery phase was followed by centrifugation of the cells for 5 minutes at 4000 rpm (3363×g), discarding of 800 μl of supernatant, and plating of 200 μl of resuspended pellet on MRS-agar containing 10 μg/ml erythromycin. The plates were incubated at 37° C. for 48 hours to allow the growth of individual colonies.

Construction of Genetic Modules in L. plantarum WCFS1

The plasmids were constructed using the DNA assembly method optimized in this study and were transformed directly into the L. plantarum WCFS1 strain. Complementary overhangs for HiFi assembly were either produced using PCR primers or synthesized as customer-specific eBlocks by IDT (Coralville, USA). Purified overlapping DNA fragments were mixed with the HiFi DNA Assembly Master Mix and were assembled as recommended in the standard reaction protocol. The assembled DNA product was then exponentially multiplied by another round of PCR using a primer pair which attaches specifically to the insert segment. 5 μl of the HiFi Assembly reaction were used as a template for PCR amplification of the assembled product (100 μl final volume). The purified PCR product was then phosphorylated using the Quick Blunting Kit. 2000 ng of the purified PCR product were mixed with 2.5 μl of 10× Quick Blunting buffer and 1 μl of enzyme mix (Milli-Q water was added to a final volume of 25 μl). The reaction was first incubated at 25° C. for 30 minutes and then at 70° C. for 10 minutes to inactivate the enzyme. Thereafter, the phosphorylated products were ligated with the aid of the enzyme T4 ligase. 6 μl of the phosphorylated DNA were mixed with 2.5 μl of 10× T4 Ligase Buffer and 1.5 μl of T4 Ligase Enzyme (Milli-Q water was added to a final volume of 25 μl). For each cloning, two ligation reactions were carried out (25 μl in each case). The respective reactions were incubated at 25° C. for 2 hours and then at 70° C. for 30 minutes to inactivate the enzyme. The ligated reactions were combined and purified. To concentrate the purified final product, three rounds of elution were carried out instead of one. Each elution was based on 10 μl of Milli-Q water. The concentration of the purified ligation product was measured using a NanoDrop Microvolume UV-Vis spectrophotometer (ThermoFisher Scientific GmbH, Germany). Lastly, 1000 ng of ligated product were transformed into L. plantarum WCFS1 electrocompetent cells.

### Microplate Reader Setup for Thermal Gradient Analysis

The bacterial cultures were cultured in 5 mL of MRS medium (supplemented with 10 μg/mL erythromycin, except for the unmodified parent strain) at 30° C. and under continuous shaking (250 rpm). The next day, the cultures were diluted to an OD₆₀₀ of 0.1 in 3 mL of fresh antibiotic-enriched MRS medium and were propagated at 30° C. and 250 rpm. At OD₆₀₀=0.4, Fisherbrand™ 0.2 mL PCR Tube Strips with flat caps (Thermo Electron LED GmbH, Germany) were filled with the cultures and were placed in a Biometra Thermocycler (Analytik Jena. GmbH, Germany). For the Pₛₚₚ-mCherry construct, 25 ng/ml Sakacin P induction peptide (SppIp) comprising 19 amino acids and having the sequence NH₂-MAGNSSNFIHKIKQIFTHR—COOH (GeneCust, France) was added to the culture and vortexed thoroughly before the aliquots were prepared. The thermal test was carried out with a temperature gradient from 31° C. to 41° C. with regular increases of 2° C. The lid temperature was set at 50° C. to prevent the evaporation of liquid and to maintain a homogeneous temperature in the spatially distributed PCR tubes. After a time interval of 18 h, the PCR strips were centrifuged in a bench mini-centrifuge (Biozym GmbH, Germany) in order to pellet the cells and discard the supernatant. The cells were then resuspended in 200 μl of 1×PBS and added to a 96-well clear-bottom microtiter plate (Corning® 96 Well Opak Black Plate, USA). The samples were then analyzed in a Microplate Reader Infinite 200 Pro (Tecan Deutschland GmbH, Germany) and both the absorption (wavelength of 600 nm) and the intensity of mCherry fluorescence (Ex_{λ}/Em_{λ}=587 nm/625 nm) were measured. The z-position and the gain settings for recording the intensity of mCherry fluorescence were set at 19 442 μm and 136, respectively. The fluorescence values were normalized to the optical density of the bacterial cells for calculation of the relative fluorescence units (RFUs) according to the formula RFU=fluorescence/OD600.

### Fluorescence Microscopy Analysis

The bacterial cultures were grown overnight in 5 mL of MRS medium (supplemented with 10 μg/mL erythromycin, except for the negative control) at 37° C. with constant shaking (250 rpm). The next day, the OD₆₀₀ of the Pₛₚₚ-mCherry construct was measured and, at OD₆₀₀=0.01, subculturing was carried out. When the Pₛₚₚ-mCherry bacterial culture reached OD₆₀₀=0.3, it was induced with 25 ng/mL SppIp, and the other constructs were subcultured in fresh medium at an OD₆₀₀ of 0.01. All the cultures were then left to grow for 18 hours under the same growth conditions (37° C., 250 rpm) to prevent any heterogeneity in the expression of promoter strength owing to different growth parameters. Thereafter, the cultures (1 mL) were harvested by centrifugation (15 700×g, 5 min, 4° C.), washed twice with 1× Dulbecco's PBS (phosphate-buffered saline) and lastly resuspended in 1 mL of 1×PBS. The suspensions (10 μL) were placed on 1.5 mm thick glass slides (Paul Marienfeld GmbH, Germany) and covered with 1.5H glass cover slips (Carl Roth GmbH, Germany). The samples were then observed under a 100× plan apochromatic oil-immersion objective lens (BZ-PA100, NA 1.45, WD 0.13 mm) of a BZ-X800 fluorescence microscope (Keyence Corporation, Illinois, USA). The mCherry signal was recorded using a BZ-X TRITC filter (model OP-87764) at an excitation wavelength of 545/25 nm and an emission wavelength of 605/70 nm with a dichroic mirror at a wavelength of 565 nm. The images were adjusted to identical brightness and contrast settings and processed using FiJi ImageJ2 software.

### Flow Cytometry Analysis

The levels of fluorescence protein expression in the strains was quantified using a Guava easyCyte BG flow cytometer (Luminex, USA). For the flow cytometry analysis, bacterial cultures subjected to the abovementioned treatment conditions were used. Bacterial suspensions (1 mL) were harvested by centrifugation at 13 000 rpm (15 700×g). The supernatant was discarded, and the pellet was resuspended in 1 mL of sterile 1× Dulbecco's PBS. The samples were then serially diluted with a dilution factor (DF) of 104 and 5000 bacterial events were recorded for analysis. The experiments were carried out in triplicate on three different days. For each analysis, the nonfluorescent wild-type strain served as negative control. To remove debris and doublets during event collection and analysis, use was made of a predefined threshold based on forward scatter (FSC) and side scatter (SSC). The fluorescence intensity of mCherry was measured by excitation with a green laser at 532 nm (100 mW) and signal analysis with the Orange G detection channel at 620/52 nm. The gain settings used for data recording were: forward scatter (FSC)—11.8; side scatter (SSC)—4 and Orange G fluorescence—1.68. The compensation control for fluorescence recording was set at 0.01 with an acquisition rate of 5 decades. Data were analyzed and displayed using Luminex GuavaSoft 4.0 software for EasyCyte.

### Construction of Plasmids Based on Toxin-Antitoxin Modules

From a literature search, the effect of the Txe/Axe (toxin-antitoxin) module from E. faecium (Grady R, Hayes F. Axe-Txe, a broad-spectrum proteic toxin-antitoxin system specified by a multidrug-resistant, clinical isolate of Enterococcus faecium. Molecular microbiology. 2003; 47(5):1419-32) was tested in L. plantarum WCFS1 in order to investigate its role in antibiotic-free plasmid retention. The tool TA Finder version 2.0 (Xie Y, Wei Y, Shen Y, Li X, Zhou H, Tai C, et al. TADB 2.0: an updated database of bacterial type II toxin-antitoxin loci. Nucleic acids research. 2018; 46(D1):D749-D53) was used to select further type II TA (toxin-antitoxin) systems in Lactobacillus genomes. The genomes of L. acidophilus, L. crispatus, L. casei, L. reuteri and L. plantarum WCFS1 were retrieved from NCBI Genome. The TA systems present in these genomes were searched using the standard parameters of TA Finder. Only TA systems annotated by NCBI BlastP were selected as test candidates. The TA systems YafQ/DinJ, HicA/HicB, HigB/HigA, MazF/MazE from L. casei, L. acidophilus and L. plantarum WCFS1 were selected for further testing and analyses.

The Axe/Txe system was amplified by PCR from the plasmid pUC-GFP-AT (Fedorec A J, Ozdemir T, Doshi A, Ho Y-K, Rosa L, Rutter J, et al. Two new plasmid post-segregational killing mechanisms for the implementation of synthetic gene networks in Escherichia coli. Iscience. 2019; 14:323-34). The DinJ/YafQ and HicA/HicB systems were synthesized as customer-specific eBlocks by IDT (Coralville, USA). HigA/HigB and MazE/MazF were amplified from the genome of L. plantarum WCFS1. The TA systems were introduced into the P_{tlpA}-mCherry plasmid, thereby yielding the plasmids P_{tlpA}-mCherry-Txe/Axe, P_{tlpA}-mCherry-YafQ/DinJ, P_{tlpA}-mCherry-HicA/HicB, P_{tlpA}mCherry-HigB/HigA, P_{tlpA}-mCherry-MazF/MazE. All these plasmids were directly cloned into L. plantarum WCFS1 using the DNA assembly method optimized in this study. For the construction of the combinatorial TA module (P_{tlpA}-mCherry Combo), the best endogenous and nonendogenous TA systems detected after 100 generations (MazF/MazE and YafQ/DinJ) were subcloned and integrated into the same plasmid in reverse orientation.

### TA-Mediated Plasmid Retention Analysis

The TA modules contained by the constructs were inoculated in 5 mL cultures containing 10 μg/mL erythromycin-enriched MRS medium and incubated overnight at 37° C. under constant shaking (250 rpm). The next day, the constructs were subcultured at an initial OD₆₀₀=0.01 in fresh MRS medium (either with or without antibiotic supplement). The bacterial cultures were incubated on 12 consecutive days with a daily growth time of 24 hours in order to ensure an average number of 8 generations per day until the final threshold of 100 generations was exceeded. Samples were prepared for flow cytometry analysis according to the abovementioned protocol. The mCherry-positive cell population directly correlated with the bacterial population containing the manipulated plasmid. The entire experiment was repeated in biological triplicates.

For qualitative analysis, the bacterial cultures grown for 100 generations without antibiotic supplement were centrifuged and resuspended in 1 ml of sterile 1× Dulbecco's PBS. The resuspended bacterial solution was diluted (DF=10⁶) and applied to MRS-agar plates with or without antibiotic supplement and incubated in a static incubator for 48 hours. The plates were then imaged using a Fluorchem Q gel documentation system (Alpha Innotech Biozym GmbH, Germany) in both the ethidium bromide channel (Ex_{λ}/Em_{λ}=300 nm/600 nm) and the Cy3 channel (Ex_{λ}/Em_{λ}=554 nm/568 nm) in order to visualize the mCherry fluorescence-producing cell population.

### Growth Rate Measurements

The influence of heterologous protein production and toxin-antitoxin modules on bacterial growth rate was investigated by culturing the bacterial cultures overnight in antibiotic-enriched MRS media at 37° C. under constant shaking (250 rpm). The next day, the bacterial cultures were subcultured into secondary cultures at an initial OD₆₀₀=0.01. After 4 hours of incubation at 37° C., the OD₆₀₀ of the cultures reached 0.1, and the cultures (200 μL) were distributed into UV STAR Flat Bottom 96 Well microtiter plates (Greiner BioOne GmbH, Germany). The 96-well assay plate was placed in the microplate reader under constant shaking at an incubation temperature of 37° C. The kinetic assay was set for recording of the absorption by the bacterial cultures at a wavelength of 600 nm at an interval of 10 minutes over a period of 18 hours. The experiment was carried out in triplicate on three independent days.

### Experimental Results

Direct Cloning into L. plantarum WCFS1

In many cases, sufficient amounts of plasmid (˜1 μg) for transformation into Lactobacillus are obtained by using shuttle vectors which can be amplified in E. coli. At the same time, it is known that certain plasmid constructs which were passaged in E. coli and contain signal peptides and repeating sequences in the gene of interest have caused mutations. This limitation was overcome by carrying out PCR-based amplification and circularization of the recombinant plasmids in order to obtain 1000 ng of circular plasmid, which was sufficient for transformation into L. plantarum WCFS1. The transformation efficiency was relatively low at 50 cfu/μg plasmid, but the genetic fidelity was >95%, since L. plantarum contains three endogenous plasmids. Since L. plantarum has three endogenous plasmids, sequencing was carried out on PCR-amplified sections of the transformed plasmid from cell lysates. This direct cloning strategy was used hereinafter for the transformation of all plasmids into L. plantarum WCFS1.

1—The tlpA Promoter from Salmonella Drives Constitutive Expression at a High Level

In the search for a heat-inducible promoter for therapeutic applications, mCherry expression was encoded downstream of the P_{tlpA} promoter from Salmonella together with the temperature-reactive TlpA repressor (Piraner D I, Abedi M H, Moser B A, Lee-Gosselin A, Shapiro M G. Tunable thermal bioswitches for in vivo control of microbial therapeutics. Nature chemical biology. 2017; 13(1):75-80; Hurme R, Berndt K D, Normark S J, Rhen M. A proteinaceous gene regulatory thermometer in Salmonella. Cell. 1997; 90(1):55-64). Whereas the repressor did not suppress gene expression (FIG. 5D), it appeared that, surprisingly, the promoter constitutively brought about a high level of protein expression with a low degree of thermal regulation (<5-fold increase from 31° C. to 39° C.) (FIG. 1A). In comparison, pR and pL, two other commonly used promoters with a temperature-reactive repressor, cI, from E. coli lambda phages, were found to be very weak and drove only low levels of mCherry expression (FIGS. 5A, 5B). Fluorescence spectroscopy showed that the strength of the P_{tlpA} promoter at 37° C. was 26 and 39 times higher than that of the P_{R} and P_{L} promoters, respectively (FIG. 5C). It is particularly noteworthy that fluorescence microscopy and analysis by flow cytometry also showed that the mCherry expression initiated by the P_{tlpA} promoter distinctly exceeds that initiated by the strongest promoters previously reported in L. plantarum—P₂₃ (Meng Q, Yuan Y, Li Y, Wu S, Shi K, Liu S. Optimization of electrotransformation parameters and engineered promoters for Lactobacillus plantarum from wine. ACS Synthetic Biology. 2021; 10(7):1728-38), P₄₈ (Rud I, Jensen P R, Naterstad K, Axelsson L. A synthetic promoter library for constitutive gene expression in Lactobacillus plantarum. Microbiology. 2006; 152(4):1011-9), Pₛₚₚ (Sørvig E, Mathiesen G, Naterstad K, Eijsink V G, Axelsson L. High-level, inducible gene expression in Lactobacillus sakei and Lactobacillus plantarum using versatile expression vectors. Microbiology. 2005; 151(7):2439-49) and P_{Tuf} (Spangler J R, Caruana J C, Phillips D A, Walper S A. Broad range shuttle vector construction and promoter evaluation for the use of Lactobacillus plantarum WCFS1 as a microbial engineering platform. Synthetic Biology. 2019; 4(1):ysz012) (FIG. 1B, FIG. 6A). At 31° C., mCherry expression was at least 2 times higher than for the other promoters, whereas at 39° C. it rose to 5 times (FIG. 1C, FIG. 6B). The constitutive promoters (P₂₃, P₄₈, P_{Tuf}) were slightly temperature-reactive, whereas the inducible promoter (Pₛₚₚ) was not (FIG. 7A). It is noteworthy that, in all cases, the expression level was greatly influenced by the spacer length between the ribosome binding site (RBS, 5′-AGGAGA-3′) and the start codon. In line with previous reports, a high level of mCherry expression was observed for a spacer length of 9 bp, and there was a distinct decrease in expression when the spacer length was reduced to 6 bp (25 times lower for P_{tlpA}) (FIG. 7B). Despite the high level of protein expression initiated by Pₜₗₚₐ with a 9 bp spacer, the growth rate of this strain at 37° C. was similar to that of the empty control strain, which indicates that, surprisingly, this overexpression of protein does not overload the cell metabolically (FIG. 1d).

The following vectors were produced and used:
promotor P_{L} (pL mcherry; SEQ ID No. 18), promotor P_{R} (pR mcherry; SEQ ID No. 19), promotor P_{TlpA} (pTlpA mcherry; SEQ ID No. 20), promotor P_{TlpA}+TlpA repressor (pTlpA A39 mcherry; SEQ ID No. 21), promotor P₂₃ (P₂₃ mcherry; SEQ ID No. 22), promotor P₄₈ (P48 mcherry; SEQ ID No. 23), promotor Pₛₚₚ (SPP mcherry; SEQ ID No. 24), promotor P_{tuf} (tuf mcherry; SEQ ID No. 25), empty vector (SEQ ID No. 32)

### 2—Toxin-Antitoxin (TA)-Based Plasmid Retention and Transient GMOs

TA systems ensure the maintenance of plasmids in a bacterial population by a postsegregational killing mechanism. They constitutively express long-lived toxins and short-lived antitoxins. As long as the plasmid is present, sufficient antitoxin is produced for neutralization of the corresponding toxin. If a daughter cell does not receive any plasmid copies on bacterial division, the antitoxin is rapidly degraded and the active toxin kills the cell. A number of natural TA systems were studied (Yamaguchi Y, Inouye M. Regulation of growth and death in Escherichia coli by toxin-antitoxin systems. Nature Reviews Microbiology. 2011; 9(11):779-90) and some of them were tested for plasma retention in E. coli with promising results (Fedorec A J, Ozdemir T, Doshi A, Ho Y-K, Rosa L, Rutter J, et al. Two new plasmid post-segregational killing mechanisms for the implementation of synthetic gene networks in Escherichia coli. Iscience. 2019; 14:323-34; Wright O, Delmans M, Stan G-B, Ellis T. GeneGuard: a modular plasmid system designed for biosafety. ACS synthetic biology. 2015; 4(3):307-16; Abedi M H, Yao M S, Mittelstein D R, Bar-Zion A, Swift M B, Lee-Gosselin A, et al. Ultrasound-controllable engineered bacteria for cancer immunotherapy. Nature communications. 2022; 13(1):1-11); research on their use in Lactobacillus has so far not been carried out. To this end, the Txe/Axe type II TA system, which comes from Enterococcus faecium and was found to ensure long-term plasmid retention in E. coli, was first investigated (Fedorec A J, Ozdemir T, Doshi A, Ho Y-K, Rosa L, Rutter J, et al. Two new plasmid post-segregational killing mechanisms for the implementation of synthetic gene networks in Escherichia coli. Iscience. 2019; 14:323-34). In this system, Txe is an endoribonuclease and Axe is the corresponding inhibitory protein. This module was added to the plasmid encoding the Pₜₗₚₐ-controlled mCherry expression (FIG. 2A) and the resultant strain was subcultured repeatedly for up to 100 generations. Plasmid retention was quantified by determining the proportion of the mCherry-expressing bacterial population with the aid of flow cytometry and agar plate colony image analysis (FIG. 8B). The sensitivity of this analysis was considerably improved by the high expression rate driven by the P_{tlpA} promoter, which allowed clear differentiation between cells with plasmid retention and those without plasmid (FIG. 2B). Such clear differentiation was not possible with other promoters such as P₂₃ because of partial overlap of the fluorescence signal with the background signal of nonfluorescent cells (FIG. 8A). In the absence of a TA system (P_{tlpA} mCherry plasmid), the proportion of plasmid-bearing bacteria decreased steadily by about 1% per generation and ended with ˜15% of the population retaining the plasmid after 100 generations (FIG. 2C). In comparison, the Txe/Axe system initially supported better retention with a plasmid loss of about 0.5% per generation for 40 generations, and then this loss accelerated to about 1.2% per generation and ended at ˜18% of the population retaining the plasmid after 100 generations. In an attempt to identify potentially more efficient TA systems, the bioinformatics tool TA Finder was used, and 4 further type II TA systems were selected—YafQ/DinJ from L. casei (Levante A, Folli C, Montanini B, Ferrari A, Neviani E, Lazzi C. Expression of DinJ-YafQ System of Lactobacillus casei group strains in response to food processing stresses. Microorganisms. 2019; 7(10):438), HigB/HigA and MazF/MazE from L. plantarum WCFS1 and HicA/HicB from L. acidophilus. All of these TA candidates are an endoribonuclease toxin which is directed at the RNA pool of the metabolically active and rapidly dividing microbial host. Similarly to the Txe/Axe module, these TA modules were added to the P_{tlpA} mCherry plasmid (FIG. 2C). The plasmid retention analysis showed that the functioning of the HigB/HigA and MazF/MazE systems was largely similar to that of Txe/Axe, but that they exhibited somewhat better retention after 100 generations (20% and 30%, respectively). HicA/HicB slowed plasmid loss to 0.5% per generation for 50 generations followed by 0.8% per generation, which led to a retention level of ˜35% after 100 generations. Lastly, it was found that YafQ/DinJ offers the best retention capacities with a plasmid loss of 0.5% per generation for 70 generations followed by 1% per generation, which leads to a degree of retention of ˜40% after 100 generations (FIG. 10A, 10B).

FIG. 10A shows the plasmid maintenance of various plasmids over 100 generations under antibiotic control. As expected, the plasmids are virtually completely maintained. FIG. 10B shows the retention under the control of the toxin-antitoxin systems. It shows a distinctly improved retention compared to the wild type. The combination of two toxin-antitoxin systems leads to another distinct improvement.

Basic studies have shown that various TA systems can cumulatively provide better plasma retention capacities (Bardaji L, Añorga M, Echeverría M, Ramos C, Murillo J. The toxic guardians-multiple toxin-antitoxin systems provide stability, avoid deletions and maintain virulence genes of Pseudomonas syringae virulence plasmids. Mobile DNA. 2019; 10(1):1-17). On this basis, the most efficient endogenous TA system from L. plantarum WCFS1 (MazF/MazE) was combined with the most efficient nonendogenous system (YafQ/DinJ). Interestingly, better plasmid retention capacities were observed with this combination, with a plasmid loss of 0.2% per generation for 50 generations and a gradual rise to 0.8% per generation thereafter, which led to a distinctly higher retention of 60% over 100 generations. In comparison, plasmids kept under antibiotic selection pressure were, as expected, maintained to an extent of >90% over 100 generations. In all the TA systems, the bacterial growth rates over 10 generations did not differ significantly from the conditions without TA or with antibiotic retention, which indicates that the toxins did not drastically impair the regular function of the cells (FIG. 9). In the TA systems, the loss of plasmid only results in the bacterium being restored to its natural, genetically unmodified probiotic status, allowing the construction of transient GMOs.

Owing to the beneficial presence of Lactobacilli in the human body, food and environment, there is considerable interest in the genetic engineering of high-level heterologous protein production for medical and industrial applications. Among the various strains, L. plantarum is one of the most intensively studied, and the search for strong promoters generally involved screening the genome of the host strain and adaptation of the promoters which allow a high level of protein expression in phylogenetically close lactic acid bacteria (FIG. 3A). Only very few studies have tested promoters of phylogenetically distant species such as P. megaterium (P_{xylA}) or E. coli (Pry of lambda phage) (Heiss S, Hörmann A, Tauer C, Sonnleitner M, Egger E, Grabherr R, et al. Evaluation of novel inducible promoter/repressor systems for recombinant protein expression in Lactobacillus plantarum. Microbial cell factories. 2016; 15(1):1-17), though the expression rates were low. Surprisingly, the promoter (P_{tlpA}) from the very phylogenetically distant gram-negative Salmonella typhimurium makes it possible to drive protein expression more strongly than the previously reported strong promoters in L. plantarum WCFS1. In Salmonella, Pₜₗₚₐ is a promoter of the σ70 sigma factors, which are involved in regulating the expression of housekeeping genes in most prokaryotes. Todt et al. (Todt T J, Wels M, Bongers R S, Siezen R S, Van Hijum S A, Kleerebezem M. Genome-wide prediction and validation of sigma70 promoters in Lactobacillus plantarum WCFS1. 2012) used a genome-wide analysis approach to identify σ70-based promoter consensus sequences in L. plantarum WCFS1 and predicted 568 promoter regions in close proximity to transcription sites (<40 nt). Their results in combination with similar analyses from other organisms (Davis M C, Kesthely C A, Franklin E A, MacLellan SR. The essential activities of the bacterial sigma factor. Canadian journal of microbiology. 2017; 63(2):89-99; Paget M S, Helmann J D. The σ70 family of sigma factors. Genome biology. 2003; 4(1):1-6) indicate that the σ70 promoter binding motifs are conserved in various genera and that divergent promoters recruiting σ70 for control of transcription in these bacterial species are possible. Multiple sequence alignment (MSA) between the key RNA polymerase σ70 proteins (RpoD) of E. coli, S. typhimurium and L. plantarum strains (FIG. 3B) revealed significant similarity between the domain 2 and domain 4 regions, which are responsible for binding to the −10 and −35 regions of the promoter during initiation of transcription.

Apart from high expression rate, the medical and industrial application of Lactobacilli requires strategies for obtaining heterologous genes in the manipulated bacteria in a cost-effective and compatible manner. Whereas genomic integration ensures stable retention of heterologous genes, plasmids offer much greater versatility and a simpler technique. In most cases, retention is ensured by providing an antibiotic resistance gene in the plasmid and cultivating the bacteria in the presence of the antibiotic. The most important antibiotic-free retention strategy for Lactobacilli is that of producing auxotrophic strains by switching off a major gene for metabolism and incorporating said gene in the plasmid. However, the need to develop auxotrophic strains by gene knockout limits the broad application of this strategy. Here, for the first time, research has been carried out on toxin-antitoxin systems for plasmid retention in Lactobacilli that do not require genome manipulation and can be easily applied to different strains without the need for external selection pressure conditions. Such TA systems were previously only tested in a few bacterial species and showed considerable retention capacities in E. coli. The results indicate that both homologous and heterologous TA systems contribute to slowing plasmid loss over multiple generations, though retention over 100 generations was only between 10% and 30%. Interestingly, the combination of two TA systems in a single plasmid led to a considerable improvement in retention, specifically up to 60% over 100 generations (FIG. 10 B). This improved cumulative effect of TA systems was observed in nature (Bardaji L, Añorga M, Echeverría M, Ramos C, Murillo J. The toxic guardians-multiple toxin-antitoxin systems provide stability, avoid deletions and maintain virulence genes of Pseudomonas syringae virulence plasmids. Mobile DNA. 2019; 10(1):1-17), but has not yet been shown in a genetically modified plasmid, especially in a combination of heterologous and homologous TA systems.

It is important to note that a single generation corresponds to doubling of bacteria, meaning that 10 generations=2¹⁰ or ˜10³ bacteria and 100 generations=2¹⁰⁰ or ˜10³⁰ bacteria from a single cell. Potential applications of Lactobacillus for live therapeutics or engineered living materials are not expected to reach such high generation numbers, either because of short application times (Watterlot L, Rochat T, Sokol H, Cherbuy C, Bouloufa I, Lefèvre F, et al. Intragastric administration of a superoxide dismutase-producing recombinant Lactobacillus casei BL23 strain attenuates DSS colitis in mice. International journal of food microbiology. 2010; 144(1):35-41; Janahi E M A, Haque S, Akhter N, Wahid M, Jawed A, Mandal R K, et al. Bioengineered intravaginal isolate of Lactobacillus plantarum expresses algal lectin scytovirin demonstrating anti-HIV-1 activity. Microbial pathogenesis. 2018; 122:1-6; Wang M, Fu T, Hao J, Li L, Tian M, Jin N, et al. A recombinant Lactobacillus plantarum strain expressing the spike protein of SARS-COV-2. International journal of biological macromolecules. 2020; 160:736-40) or because of external growth restrictions (Bhusari S, Sankaran S, del Campo A. Regulating bacterial behavior within hydrogels of tunable viscoelasticity. bioRxiv. 2022). Therefore, the >90% retention rates provided by the Combo-TA system for up to 40 generations should be more than sufficient for these applications. Since the loss of plasmid only restores the bacteria to their probiotic non-GMO status, such transient GMOs may even be desirable for such applications. Accordingly, the GMO lifetime of these organisms may be adjusted by varying the TA system used. This concept has been used to introduce a new metric, G₅₀, for characterization of such transient GMOs. The G₅₀ value corresponds to the generation in which half of the population of a strain has lost its plasmid. As shown in FIG. 4, a G₅₀ of 50 generations for the condition “No TA” can be set as far as 110 generations (extrapolated) for the Combo system. Further research into additional TA systems in future studies will contribute to greater fine-tuning of retention lifetimes and may even lead to virtually perfect retention, as achieved in E. coli by the Txe/Axe system (Fedorec A J, Ozdemir T, Doshi A, Ho Y-K, Rosa L, Rutter J, et al. Two new plasmid post-segregational killing mechanisms for the implementation of synthetic gene networks in Escherichia coli. Iscience. 2019; 14:323-34). These G₅₀ values are expected to depend on culture parameters and environmental factors, and so they may also become a useful benchmark for assessing the natural and industrial conditions under which Lactobacilli are growing and functioning.

The following vectors were used:
P_{TlpA}+Txe/Axe (pTlpA mcherry Txe/Axe; SEQ ID No. 26), P_{Tlpa}+YafQ/DinJ (pTlpA mcherry YafQ/DinJ; SEQ ID No. 27), P_{TlpA}+HigB/HigA (pTlpA mcherry HigB/HigA; SEQ ID No. 28), P_{TlpA}+MazF/MazE (pTlpA mcherry MazF/MazE; SEQ ID No. 29), P_{TlpA}+HicA/HicB (pTlpA mcherry HicA/HicB; SEQ ID No. 30), P_{TlpA}+MazF/MazE+YafQ/DinJ (pTlpA mcherry Combo; SEQ ID No. 31). In the figures and in the description, the vectors are also referred to without the indication “pTlpA mcherry”, e.g., HigB/HigA or Combo. TlpA is also specified as tlpA.

All sequences and their names are also specified in Table 2.

FIG. 11 showed the schematic illustration of the vector with origin of replication (ori); eryR: erythromycin resistance; toxin-antitoxin. The arrows represent promoters and T represents terminators. Antibiotic resistance is optional. The toxin-antitoxin system has its own promoter. The order of toxin-antitoxin may also be reversed. The vector may also have multiple toxin-antitoxin systems.

TABLE 1

| Seq | Pro- | |
|---|---|---|
| ID | moter | Sequence (5′-3′) |
| 1 | P_{tlpA} | tttaatttgtttgttagttagtttatt |
| | | tgttggtttgttt-gtgttataatat |
| 2 | P_{L} | ttgacataaataccactggcggtgatact |
| 3 | P_{R} | ttgactattttacctctggcggtgataa |
| 4 | P_{Tuf} | tctgtttacaaatcagattaggctatata |
| | | taatatttaagga |
| 5 | Pₛₚₚ | gcccatattaacgtttaaccgataaagtt |
| | | gaacgttaa-tatttttttt |
| 6 | P₄₈ | agttgttgacatggaacgaggaatgtgat |
| | | aatctgtgagt |
| 7 | P₂₃ | ctgatgacaaaaagagaaaattttgataa |
| | | aatagtctta-gaattaaattaaaaa |

TABLE 2

| SEQ ID | | |
|---|---|---|
| No. | Name | Length (bp) |
| 1 | PtlpA | 52 |
| 2 | PL | 29 |
| 3 | PR | 28 |
| 4 | PTuf | 42 |
| 5 | Pspp | 48 |
| 6 | P48 | 40 |
| 7 | P23 | 54 |
| 8 | Txe | 258 |
| 9 | Axe | 270 |
| 10 | YafQ | 303 |
| 11 | DinJ | 270 |
| 12 | HicA | 210 |
| 13 | HicB | 342 |
| 14 | HigB | 228 |
| 15 | HigA | 255 |
| 16 | MazF | 348 |
| 17 | MazE | 219 |
| 18 | pL mcherry | 3535 |
| 19 | pR mcherry | 3535 |
| 20 | pTlpA mcherry | 6084 |
| 21 | pTlpA A39 mcherry | 7330 |
| 22 | P23 mcherry | 3535 |
| 23 | P48 mcherry | 3533 |
| 24 | SPP mcherry | 6161 |
| 25 | tuf mcherry | 3535 |
| 26 | pTlpA mcherry Txe/Axe | 9432 |
| 27 | pTlpA mcherry YafQ/DinJ | 5208 |
| 28 | pTlpA mcherry HigB/HigA | 5196 |
| 29 | pTlpA mcherry MazF/MazE | 5182 |
| 30 | pTlpA mcherry HicA/HicB | 4992 |
| 31 | pTlpA mcherry Combo | 5643 |
| 32 | vector_emtpy | 2675 |

### LITERATURE CITED

Rud I, Jensen P R, Naterstad K, Axelsson L. A synthetic promoter library for constitutive gene expression in Lactobacillus plantarum. Microbiology. 2006; 152(4):1011-9.
Heiss S, Hörmann A, Tauer C, Sonnleitner M, Egger E, Grabherr R, et al. Evaluation of novel inducible promoter/repressor systems for recombinant protein expression in Lactobacillus plantarum. Microbial cell factories. 2016; 15(1):1-17.
Sørvig E, Mathiesen G, Naterstad K, Eijsink V G, Axelsson L. High-level, inducible gene expression in Lactobacillus sakei and Lactobacillus plantarum using versatile expression vectors. Microbiology. 2005; 151(7):2439-49.
Mathiesen G, Sveen A, Brurberg M B, Fredriksen L, Axelsson L, Eijsink V G. Genome-wide analysis of signal peptide functionality in Lactobacillus plantarum WCFS1. BMC genomics. 2009; 10(1):1-13.
Piraner D I, Abedi M H, Moser B A, Lee-Gosselin A, Shapiro M G. Tunable thermal bioswitches for in vivo control of microbial therapeutics. Nature chemical biology. 2017; 13(1):75-80.
Fedorec A J, Ozdemir T, Doshi A, Ho Y-K, Rosa L, Rutter J, et al. Two new plasmid post-segregational killing mechanisms for the implementation of synthetic gene networks in Escherichia coli. Iscience. 2019; 14:323-34.
Grady R, Hayes F. Axe-Txe, a broad-spectrum proteic toxin-antitoxin system specified by a multidrug-resistant, clinical isolate of Enterococcus faecium. Molecular microbiology. 2003; 47(5):1419-32.
Xie Y, Wei Y, Shen Y, Li X, Zhou H, Tai C, et al. TADB 2.0: an updated database of bacterial type II toxin-antitoxin loci. Nucleic acids research. 2018; 46(D1):D749-D53.
Hurme R, Berndt K D, Normark S J, Rhen M. A proteinaceous gene regulatory thermometer in Salmonella. Cell. 1997; 90(1):55-64.
Meng Q, Yuan Y, Li Y, Wu S, Shi K, Liu S. Optimization of electrotransformation parameters and engineered promoters for Lactobacillus plantarum from wine. ACS Synthetic Biology. 2021; 10(7):1728-38.
Spangler J R, Caruana J C, Phillips D A, Walper S A. Broad range shuttle vector construction and promoter evaluation for the use of Lactobacillus plantarum WCFS1 as a microbial engineering platform. Synthetic Biology. 2019; 4(1):ysz012.
Yamaguchi Y, Inouye M. Regulation of growth and death in Escherichia coli by toxin-antitoxin systems. Nature Reviews Microbiology. 2011; 9(11):779-90.
Wright O, Delmans M, Stan G-B, Ellis T. GeneGuard: a modular plasmid system designed for biosafety. ACS synthetic biology. 2015; 4(3):307-16.
Abedi M H, Yao M S, Mittelstein D R, Bar-Zion A, Swift M B, Lee-Gosselin A, et al. Ultrasound-controllable engineered bacteria for cancer immunotherapy. Nature communications. 2022; 13(1):1-11.
Levante A, Folli C, Montanini B, Ferrari A, Neviani E, Lazzi C. Expression of DinJ-YafQ System of Lactobacillus casei group strains in response to food processing stresses. Microorganisms. 2019; 7(10):438.
Bardaji L, Añorga M, Echeverría M, Ramos C, Murillo J. The toxic guardians-multiple toxin-antitoxin systems provide stability, avoid deletions and maintain virulence genes of Pseudomonas syringae virulence plasmids. Mobile DNA. 2019; 10(1):1-17.
Todt T J, Wels M, Bongers R S, Siezen R S, Van Hijum S A, Kleerebezem M. Genome-wide prediction and validation of sigma70 promoters in Lactobacillus plantarum WCFS1. 2012.
Davis M C, Kesthely C A, Franklin E A, MacLellan S R. The essential activities of the bacterial sigma factor. Canadian journal of microbiology. 2017; 63(2):89-99.
Paget M S, Helmann J D. The σ70 family of sigma factors. Genome biology. 2003; 4(1):1-6.
Bhusari S, Sankaran S, del Campo A. Regulating bacterial behavior within hydrogels of tunable viscoelasticity. bioRxiv. 2022.
Singh G. et al. Current Research in Microbial Sciences 2, 2021, 100047, Bacterial toxin-antitoxin modules: classifica-tion, functions, and association with persistence
Gerdes, K., Bech, F. W., Jørgensen, S. T., Løbner-Olesen, A., Rasmussen, P. B., Atlung, T., Boe, L., Karlstrom, O., Molin, S., von Meyenburg, K., 1986. Mechanism of postsegregational killing by the hok gene product of the parB system of plasmid R1 and its homology with the relF gene product of the E. coli relB operon. EMBO J 5 (8), 2023-2029.

1. An expression cassette for expression in Lactobacillus comprising a promoter operatively linked to a nucleic acid encoding a protein to be expressed, the promoter being substantially identical to the P_{tlpA} promoter.

2. The expression cassette, wherein the promoter has a sequence substantially identical to sequence ID No. 1.

3. The expression cassette as claimed in claim 1, wherein the expression cassette is operatively linked to at least one toxin-antitoxin system.

4. The expression cassette as claimed in claim 3, wherein the expression cassette is operatively linked to at least two toxin-antitoxin systems.

5. The expression cassette as claimed in claim 3, wherein the at least one toxin-antitoxin system is selected from the group consisting of Txe (SEQ ID No. 8)/Axe (SEQ ID No. 9), YafQ (SEQ ID No. 10)/DinJ (SEQ ID No. 11), HicA (SEQ ID No. 12)/HicB (SEQ ID No. 13), HigB (SEQ ID No. 14)/HigA (SEQ ID No. 15) and MazF (SEQ ID No. 16)/MazE (SEQ ID No. 17).

6. A vector comprising the expression cassette as claimed in claim 1.

7. A Lactobacillus comprising at least one expression cassette as claimed in claim 1.

8. A Lactobacillus comprising at least one vector as claimed in claim 6.

9. The use of the promoter of the expression cassette as claimed in claim 1 in Lactobacillus.
